(19) United States
(12) Patent Application Publication
(10) Pub. No.: US 2002/0164654 A1
(43) Pub. Date: Nov. 7, 2002

(54) MAGE antigenic peptides which bind HLA-B35 and HLA-B44

(76) Inventors: Rosalie Luiten, Amsterdam (NL); Thierry Boon-Falleur, Brussels (BE); Pierre Van der Bruggen, Brussels (BE); Vincent Stroobant, Brussels (BE); Nathalie Demotte, Brussels (BE); Erwin Schultz, Erlangen (DE)

Correspondence Address: John R. Van Amsterdam, Wolf, Greenfield & Sacks, P.C., Federal Reserve Plaza, 600 Atlantic Avenue, Boston, MA, 02210-2211, US

(21) Appl. No.: 09/766,889

(22) Filed: Jan. 19, 2001

Related U.S. Application Data
(60) Provisional application No. 60/177,242, filed on Jan. 20, 2000.
(60) Provisional application No. 60/243,212, filed on Oct. 25, 2000.

(51) Int. Cl. G01N 33/53; G01N 33/567

(52) U.S. Cl. 435/7.2

(57) ABSTRACT

The invention provides antigenic peptides derived from MAGE-A1 polypeptides and presented by HLA-B35 and HLA-B44 molecules. Antigenic peptides derived from MAGE-A3 polypeptides and presented by HLA-B35 molecules also are provided. Methods for diagnosis and treatment which involve the polypeptides also are provided.

### RELATED APPLICATIONS

This application claims priority under 35 U.S.C. §119 from U.S. provisional application serial No. 60/177,242, filed Jan. 20, 2000, and from from U.S. provisional application serial No. 60/243,212, filed Oct. 25, 2000.

### FIELD OF THE INVENTION

This invention relates to fragments of the MAGE tumor associated gene products which bind to and are presented to T lymphocytes by HLA-B35 and HLA-B44 molecules. The peptides, nucleic acid molecules which code for such peptides, as well as related antigen presenting cells and CD8⁺ T lymphocytes, are useful, inter alia, in diagnostic and therapeutic contexts.

### BACKGROUND OF THE INVENTION

The phenotypic changes which distinguish a tumor cell from its normal counterpart are often the result of one or more changes to the genome of the cell. The genes which are expressed in tumor cells, but not in normal counterparts, can be termed “tumor associated” genes. These tumor associated genes are markers for the tumor phenotype. The expression of tumor associated genes can also be an essential event in the process of tumorigenesis.

Typically, the host recognizes as foreign the tumor associated genes which are not expressed in normal non-tumorigenic cells. Thus, the expression of tumor associated genes can provoke an immune response against the tumor cells by the host. Tumor associated genes can also be expressed in normal cells within certain tissues without provoking an immune response. In such tissues, expression of the gene and/or presentation of an ordinarily immunologically recognizable fragment of the protein product on the cell surface may not provoke an immune response because the immune system does not “see” the cells inside these immunologically privileged tissues. Examples of immunologically privileged tissues include brain and testis.

The discovery of tumor associated expression of a gene provides a means of identifying a cell as a tumor cell. Diagnostic compounds can be based on the tumor associated gene, and used to determine the presence and location of tumor cells. Further, when the tumor associated gene contributes to an aspect of the tumor phenotype (e.g., unregulated growth or metastasis), the tumor associated gene can be used to provide therapeutics such as antisense nucleic acids which can reduce or substantially eliminate expression of that gene, thereby reducing or substantially eliminating the phenotypic aspect which depends on the expression of the particular tumor associated gene.

The process by which the mammalian immune system recognizes and reacts to foreign or alien materials is a complex one. An important facet of the system is the T cell response. This response requires that T cells recognize and interact with complexes of cell surface molecules, referred to as human leukocyte antigens (“HLA”), or major histocompatibility complexes (“MHCs”), and peptides. The peptides are derived from larger molecules which are processed by the cells which also present the HLA/MHC molecule. See in this regard Male et al., Advanced Immunology (J.P. Lipincott Company, 1987), especially chapters 6-10. The interaction of T cells and complexes of HLA/peptide is restricted, requiring a T cell specific for a particular combination of a HLA molecule and a peptide. If a specific T cell is not present, there is no T cell response even if its partner complex is present. Similarly, there is no response if the specific complex is absent, but the T cell is present. The mechanism is involved in the immune system's response to foreign materials, in autoimmune pathologies, and in responses to cellular abnormalities. Much work has focused on the mechanisms by which proteins are processed into the HLA binding peptides. See, in this regard, Barinaga, Science 257: 880, 1992; Fremont et al., Science 257: 919, 1992; Matsumura et al., Science 257: 927, 1992; Latron et al., Science 257: 964, 1992.

The mechanism by which T cells recognize cellular abnormalities has also been implicated in cancer. For example, in PCT application PCT/US92/04354, filed May 22, 1992, published on Nov. 26, 1992, and incorporated by reference, a family of genes is disclosed, which are processed into peptides which, in turn, are expressed on cell surfaces, which can lead to lysis of the tumor cells by specific CTLs. The genes are said to code for “tumor rejection antigen precursors” or “TRAP” molecules, and the peptides derived therefrom are referred to as “tumor rejection antigens” or “TRAs”. See Traversari et al., J Exp. Med. 176:1453-1457, 1992; van der Bruggen et al., Science 254: 1643,1991; De Plaen et al., Immunogenetics 40:360-369, 1994 and U.S. Pat. No. 5,342,774 for further information on this family of genes.

In U.S. Pat. No. 5,405,940, the disclosure of which is incorporated by reference, nonapeptides are taught which are presented by the HLA-A1 molecule. The reference teaches that given the known specificity of particular peptides for particular HLA molecules, one should expect a particular peptide to bind one HLA molecule, but not others. This is important, because different individuals possess different HLA phenotypes. As a result, while identification of a particular peptide as being a partner for a specific HLA molecule has diagnostic and therapeutic ramifications, these are only relevant for individuals with that particular HLA phenotype. There is a need for further work in the area, because cellular abnormalities are not restricted to one particular HLA phenotype, and targeted therapy requires some knowledge of the phenotype of the abnormal cells at issue.

In U.S. Pat. 5,629,166, incorporated by reference, the fact that the MAGE-A1 expression product is processed to a second TRA is disclosed. This second TRA is presented by HLA-Cw16 molecules, also known as HLA-C*1601. The disclosure shows that a given TRAP can yield a plurality of TRAs.

In U.S. Pat. 5,487,974, incorporated by reference herein, tyrosinase is described as a tumor rejection antigen precursor. This reference discloses that a molecule which is produced by some normal cells (e.g., melanocytes), is processed in tumor cells to yield a tumor rejection antigen that is presented by HLA-A2 molecules.

In U.S. Pat. No. 5,620,886, incorporated herein by reference in its entirety, a second TRA, not derived from tyrosinase is taught to be presented by HLA-A2 molecules. The TRA is derived from a TRAP, but is coded for by a known MAGE gene. This disclosure shows that a particular HLA molecule may present TRAs derived from different sources.

Additional TRAPs are disclosed in U.S. Pat. Nos. 5,571,711, 5,610,013, 5,587,289 and 5,589,334, as well as PCT publication WO96/10577. The TRAPs are processed to tumor rejection antigens, which are presented by a variety of HLA molecules.

The MAGE-encoded antigens that are recognized by cytolytic T lymphocytes (CTL) are shared by many tumors and are strictly tumor-specific. Clinical trials involving therapeutic vaccination of cancer patients with MAGE antigenic peptides or proteins are in progress. To increase the range of patients eligible for therapy with peptides, it is important to identify additional MAGE epitopes. There exist many patients who would benefit from therapy which includes additional antigenic MAGE peptides, either because the patient's tumor does not express previously known antigenic peptides, or because the patient does not express the appropriate HLA molecule. Accordingly, there is a need for the identification of additional tumor rejection antigens which are presented by MHC class I molecules and recognized by CD8⁺ lymphocytes.

### SUMMARY OF THE INVENTION

It now has been discovered that human MAGE genes encode tumor rejection antigens presented by HLA-B35 and HLA-B44, which previously were not known to present MAGE peptides. Peptides derived from the MAGE-A1 polypeptide (SEQ ID NO:2, encoded by SEQ ID NO:1), when presented by an antigen presenting cell having a HLA-B35 or HLA-B44 molecule, effectively induce the activation and proliferation of CD8⁺ cytotoxic T lymphocytes. Likewise, it has been discovered that peptides derived from the human MAGE-A3 polypeptide (SEQ ID NO:55, encoded by SEQ ID NO:54), when presented by an antigen presenting cell having a HLA-B35 molecule, effectively induce the activation and proliferation of CD8⁺ cytotoxic T lymphocytes.

According to one aspect of the invention, methods for diagnosing a disorder characterized by expression of MAGE-A1 in a subject typed as HLA-B35 positive are provided. The methods include contacting a biological sample isolated from the subject with an agent that is specific for a MAGE-A1 HLA binding peptide which comprises SEQ ID NO:10, and determining the interaction between the agent and the MAGE-A1 HLA binding peptide as a determination of the disorder. In some embodiments, the MAGE-A1 HLA binding peptide is selected from the group consisting of (i) peptides which consist of a fragment of the amino acid sequence of SEQ ID NO:2, (ii) peptides which comprise the amino acid sequence of SEQ ID NO:8, and (iii) functional variants of the peptides of (i) and (ii).

According to another aspect of the invention, methods for diagnosing a disorder characterized by expression of MAGE-A1 in a subject typed as HLA-B44 positive are provided. The methods include contacting a biological sample isolated from the subject with an agent that is specific for a MAGE-A1 HLA binding peptide which comprises SEQ ID NO:53, and determining the interaction between the agent and the MAGE-A1 HLA binding peptide as a determination of the disorder. In some embodiments, the MAGE-A1 HLA binding peptide is selected from the group consisting of (i) peptides which consist of a fragment of the amino acid sequence of SEQ ID NO:2, (ii) peptides which comprise the amino acid sequence of SEQ ID NO:14, and (iii) functional variants of the peptides of (i) and (ii).

Accord to yet another aspect of the invention, methods for diagnosing a disorder characterized by expression of a complex of a MAGE-A1 HLA-B35 or HLA-B44 binding peptide and a HLA-B35 or HLA-B44 molecule are provided. The methods include contacting a biological sample isolated from a subject suspected of having the disorder with an agent that binds the complex; and determining binding between the complex and the agent as a determination of the disorder. In some embodiments, the MAGE-A1 HLA-B35 binding peptide is selected from the group consisting (i) peptides which consist of a fragment of the amino acid sequence of SEQ ID NO:2, (ii) peptides which comprise the amino acid sequence of SEQ ID NO:10, and (iii) functional variants of the peptides of (i) and (ii). In other embodiments, the MAGE-A1 HLA-B44 binding peptide is selected from the group consisting (i) peptides which consist of a fragment of the amino acid sequence of SEQ ID NO:2, (ii) peptides which comprise the amino acid sequence of SEQ ID NO:53, and (iii) functional variants of the peptides of (i) and (ii).

Methods for enriching selectively a population of T lymphocytes with T lymphocytes specific for a MAGE-A1 HLA binding peptide are provided in accordance with another aspect of the invention. The methods include contacting a source of T lymphocytes which contains a population of T lymphocytes with an agent presenting a complex of a MAGE-A1 HLA-B35 binding peptide, preferably comprising SEQ ID NO:10, and a HLA-B35 molecule or a complex of a MAGE-A1 HLA-B44 binding peptide, preferably comprising SEQ ID NO:53, and a HLA-B44 molecule, in an amount sufficient to selectively enrich the population of T lymphocytes with the T lymphocytes specific for one of the complexes. In some embodiments, the agent is selected from the group consisting of an antigen presenting cell which expresses a HLA-B35 molecule contacted with a MAGE-A1 protein or a HLA binding fragment thereof which comprises SEQ ID NO:10, and an antigen presenting cell which expresses a HLA-B44 molecule contacted with a MAGE-A1 protein or a HLA binding fragment thereof which comprises SEQ ID NO:53. In other embodiments, the MAGE-A1 HLA-B35 binding peptide is selected from the group consisting of (i) SEQ ID NO:8, SEQ ID NO:9 and SEQ ID NO:10, and (ii) functional variants of the peptides of (i). In still other embodiments, the MAGE-A1 HLA-B44 binding peptide is selected from the group consisting of (i) SEQ ID NO:12 and SEQ ID NO:14, and (ii) functional variants of the peptides of (i).

According to a further aspect of the invention, methods for treating a subject, typed as HLA-B35 or HLA-B44 positive, having a disorder characterized by expression of MAGE-A1, are provided. The methods include administering to the subject an amount of a MAGE-A1 HLA-B35 binding peptide, preferably comprising SEQ ID NO:10, or a MAGE-A1 HLA-B44 binding peptide, preferably comprising SEQ ID NO:53, sufficient to ameliorate the disorder. In some embodiments, the MAGE-A1 HLA-B35 binding peptide is selected from the group consisting of (i) peptides which consist of a fragment of the amino acid sequence of SEQ ID NO:2, (ii) peptides which comprise the amino acid sequence of SEQ ID NO:8, and (iii) functional variants of the peptides of (i) and (ii). In certain preferred embodiments, the methods also include administering to the subject at least one isolated HLA binding peptide selected from the group consisting of (1) MAGE-A1 HLA class I binding peptides other than peptides comprising SEQ ID NO:8, (2) MAGE-A1 HLA class II binding peptides, and (3) MAGE-A1 HLA class I or class II binding peptide of a non-MAGE-A1 tumor antigen, in an amount sufficient to ameliorate the disorder.

In other embodiments, the MAGE-A1 HLA-B44 binding peptide is selected from the group consisting of (i) peptides which consist of a fragment of the amino acid sequence of SEQ ID NO:2, (ii) peptides which comprise the amino acid sequence of SEQ ID NO:14, and (iii) functional variants of the peptides of (i) and (ii). In certain preferred embodiments, the methods also include administering to the subject at least one isolated HLA binding peptide selected from the group consisting of (1) MAGE-A1 HLA class I binding peptides other than peptides comprising SEQ ID NO:14, (2) MAGE-A1 HLA class II binding peptides, and (3) MAGE-A1 HLA class I or class II binding peptide of a non-MAGE-A1 tumor antigen, in an amount sufficient to ameliorate the disorder.

According to still another aspect of the invention, additional methods are provided for treating a subject, typed as HLA-B35 or HLA-B44 positive, having a disorder characterized by expression of MAGE-A1. The methods include administering to the subject an amount of an agent which enriches selectively in the subject the presence of complexes of a HLA-B35 molecule and a MAGE-A1 HLA-B35 binding peptide, preferably comprising SEQ ID NO:10, or a HLA-B44 molecule and a MAGE-A1 HLA-B44 binding peptide, preferably comprising SEQ ID NO:53, in an amount sufficient to ameliorate the disorder. In preferred embodiments, the agent comprises a MAGE-A1 HLA-B35 binding peptide selected from the group consisting of (i) peptides which consist of a fragment of the amino acid sequence of SEQ ID NO:2, (ii) peptides which comprise the amino acid sequence of SEQ ID NO:8, and (iii) functional variants of the peptides of (i) and (ii), or comprises a MAGE-A1 HLA-B44 binding peptide selected from the group consisting of (i) peptides which consist of a fragment of the amino acid sequence of SEQ ID NO:2, (ii) peptides which comprise the amino acid sequence of SEQ ID NO:14, and (iii) functional variants of the peptides of (i) and (ii).

Additional methods for treating a subject, typed as HLA-B35 or HLA-B44 positive, having a disorder characterized by expression of MAGE-A1, are provided according to yet another aspect of the invention. The methods include administering to the subject an amount of autologous T lymphocytes sufficient to ameliorate the disorder, wherein the T lymphocytes are specific for complexes of a HLA-B35 molecule and a MAGE-A1 HLA-B35 binding peptide, preferably comprising SEQ ID NO:10, or a HLA-B44 molecule and a MAGE-A1 HLA-B44 binding peptide, preferably comprising SEQ I) NO:53. In preferred embodiments, the agent comprises a MAGE-A1 HLA-B35 binding peptide selected from the group consisting of (i) peptides which consist of a fragment of the amino acid sequence of SEQ ID NO:2, (ii) peptides which comprise the amino acid sequence of SEQ ID NO:8, and (iii) functional variants of the peptides of (i) and (ii), or comprises a MAGE-A1 HLA-B44 binding peptide selected from the group consisting of (i) peptides which consist of a fragment of the amino acid sequence of SEQ ID NO:2, (ii) peptides which comprise the amino acid sequence of SEQ ID NO:14, and (iii) functional variants of the peptides of (i) and (ii).

According to still another aspect of the invention, methods for identifying functional variants of a MAGE-A1 HLA-B35 or HLA-B44 binding peptide are provided. The methods include providing (1) a MAGE-A1 HLA-B35 binding peptide, preferably comprising SEQ ID NO:10, and a HLA-B35 molecule, or (2) a MAGE-A1 HLA-B44 binding peptide, preferably comprising SEQ ID NO:53, and a HLA-B44 molecule, and a T cell which is stimulated by the MAGE-A1 HLA binding peptide presented by the HLA-B35 or HLA-B44 molecule. The methods also include mutating a first amino acid residue of the MAGE-A1 HLA-B35 or HLA-B44 binding peptide to prepare a variant peptide; and determining the binding of the variant peptide to the HLA-B35 or HLA-B44 molecule or the stimulation of the T cell, wherein binding of the variant peptide to the HLA binding molecule or stimulation of the T cell by the variant peptide presented by the HLA binding molecule indicates that the variant peptide is a functional variant.

In certain embodiments, the MAGE-A1 HLA-B35 binding peptide is selected from the group consisting of (i) peptides which consist of a fragment of the amino acid sequence of SEQ ID NO:2, (ii) peptides which comprise the amino acid sequence of SEQ ID NO:8. In other embodiments, the MAGE-A1 HLA-B44 binding peptide is selected from the group consisting of (i) peptides which consist of a fragment of the amino acid sequence of SEQ ID NO:2, (ii) peptides which comprise the amino acid sequence of SEQ ID NO:14.

In preferred embodiments, the methods further include the step of comparing the stimulation of the T cell by the MAGE-A1 HLA binding peptide and the stimulation of the T cell by the functional variant as a determination of the effectiveness of the stimulation of the T cell by the functional variant.

Isolated functional variants of a MAGE-A1 HLA-B35 or HLA-B44 binding peptide identified by the foregoing methods also are provided.

According to yet another aspect of the invention, expression vectors encoding the MAGE-A1 HLA-B35 and/or the HLA-B44 binding peptides are provided. The expression vectors which encode HLA-B35 binding peptide preferably include a nucleotide sequence which encodes an amino acid sequence selected from the group consisting of SEQ ID NO:8, SEQ ID NO:9, and SEQ ID NO:10, operably linked to a promoter, and optionally also include a nucleotide sequence which encodes the amino acid sequence of a HLA-B*35 molecule, operably linked to a promoter. The expression vectors which encode HLA-B35 binding peptide preferably include a nucleotide sequence which encodes an amino acid sequence selected from the group consisting of SEQ ID NO:12, SEQ ID NO:14 and SEQ ID NO:53, operably linked to a promoter, and optionally also include a nucleotide sequence which encodes the amino acid sequence of a HLA-B*44 molecule operably linked to a promoter.

Also provided by the invention are host cells transfected or transformed with foregoing expression vectors. Methods for producing the MAGE-A1 HLA binding peptides and complexes of the HLA binding peptides and HLA molecules by culturing the host cells and optionally isolating the expressed proteins also are provided.

According to yet another aspect of the invention, isolated T lymphocytes which selectively bind a complex of a HLA-B35 molecule and a MAGE-A1 HLA binding peptide, preferably one which comprises the amino acid sequence of SEQ ID NO:10, are provided. In certain embodiments, the MAGE-A1 HLA binding peptide is selected from the group consisting of (i) peptides which consist of a fragment of the amino acid sequence of SEQ ID NO:2, (ii) peptides which comprise the amino acid sequence of SEQ ID NO:8, and (iii) functional variants of the peptides of (i) and (ii).

Also provided by the invention are isolated T lymphocytes which selectively bind a complex of a HLA-B44 molecule and a MAGE-A1 HLA binding peptide, preferably one which comprises the amino acid sequence of SEQ ID NO:53. In certain embodiments, the MAGE-A1 HLA binding peptide is selected from the group consisting of (i) peptides which consist of a fragment of the amino acid sequence of SEQ ID NO:2, (ii) peptides which comprise the amino acid sequence of SEQ ID NO:14, and (iii) functional variants of the peptides of (i) and (ii).

According to another aspect of the invention, isolated antigen presenting cells are provided. The isolated antigen presenting cells include a complex of a HLA-B35 molecule and a MAGE-A1 HLA binding peptide, preferably one which comprises the amino acid sequence of SEQ ID NO:10. In certain embodiments, the MAGE-A1 HLA binding peptide is selected from the group consisting of (i) peptides which consist of a fragment of the amino acid sequence of SEQ ID NO:2, (ii) peptides which comprise the amino acid sequence of SEQ ID NO:8, and (iii) functional variants of the peptides of (i) and (ii). Also provided are isolated antigen presenting cells which include a complex of a HLA-B44 molecule and a MAGE-A1 HLA binding peptide, preferably one which comprises the amino acid sequence of SEQ ID NO:53. In certain embodiments, the MAGE-A1 HLA binding peptide is selected from the group consisting of (i) peptides which consist of a fragment of the amino acid sequence of SEQ ID NO:2, (ii) peptides which comprise the amino acid sequence of SEQ ID NO:14, and (iii) functional variants of the peptides of (i) and (ii).

Vaccine compositions including the foregoing MAGE-A1 HLA binding peptides, complexes of HLA molecules and HLA binding peptides, combinations of MAGE-A1 HLA binding peptides and non-MAGE-A1 HLA binding peptides, expression vectors, T lymphocytes and/or antigen presenting cells also are provided according to the invention. The vaccine compositions preferably include a pharmaceutically acceptable carrier. In preferred embodiments, the vaccine compositions also include an adjuvant.

According to yet another aspect of the invention, methods for identifying a candidate mimetic of a MAGE-A1 HLA-B35 or HLA-B44 binding peptide are provided. The methods include providing a HLA-B35 or HLA-B44 molecule, contacting the HLA molecule with a test molecule, and determining the binding of the test molecule to the HLA molecule, wherein a test molecule which binds to the HLA molecule is a candidate mimetic of the MAGE-A1 HLA binding peptide. In certain preferred embodiments, the methods include contacting the HLA molecule with a HLA-B35 binding molecule comprising the amino acid sequence of SEQ ID NO:8, and determining the binding of the HLA binding molecule to the HLA molecule in the presence and the absence of the test molecule. In other preferred embodiments, the methods include contacting the HLA molecule with a HLA-B44 binding molecule comprising the amino acid sequence of SEQ ID NO:53, and determining the binding of the HLA binding molecule to the HLA molecule in the presence and the absence of the test molecule.

In additional preferred embodiments, the methods include forming a complex of the HLA molecule and the candidate mimetic, contacting the complex with a T cell which binds to a complex of a HLA molecule and the MAGE-A1 HLA binding peptide, and assaying activation of the T cell. Preferably activation of the T cell is indicated by a property selected from the group consisting of proliferation of the T cell, interferon-γ production by the T cell, tumor necrosis factor production by the T cell, and cytolysis of a target cell by the T cell.

Isolated mimetics of a MAGE-A1 HLA-B35 or HLA-B44 binding peptide identified by the foregoing methods also are provided.

According to another aspect of the invention, methods for diagnosing a disorder characterized by expression of MAGE-A3 in a subject typed as HLA-B35 positive are provided. The methods include contacting a biological sample isolated from the subject with an agent that is specific for a MAGE-A3 HLA binding peptide which comprises SEQ ID NO:56, and determining the interaction between the agent and the MAGE-A3 HLA binding peptide as a determination of the disorder. In certain embodiments, the MAGE-A3 HLA binding peptide is selected from the group consisting of (i) peptides which consist of a fragment of the amino acid sequence of SEQ ID NO:55, (ii) peptides which comprise the amino acid sequence of SEQ ID NO:59, and (iii) functional variants of the peptides of (i) and (ii).

Also provided according to the invention are methods for diagnosing a disorder characterized by expression of a complex of a MAGE-A3 HLA-B35 binding peptide and a HLA-B35 molecule. The methods include contacting a biological sample isolated from a subject suspected of having the disorder with an agent that binds the complex; and determining binding between the complex and the agent as a determination of the disorder. In some embodiments the MAGE-A3 HLA-B35 binding peptide is selected from the group consisting (i) peptides which consist of a fragment of the amino acid sequence of SEQ ID NO:55, (ii) peptides which comprise the amino acid sequence of SEQ ID NO:56, and (iii) functional variants of the peptides of (i) and (ii).

In another aspect of the invention, methods for enriching selectively a population of T lymphocytes with T lymphocytes specific for a MAGE-A3 HLA binding peptide are provided. The methods include contacting a source of T lymphocytes which contains a population of T lymphocytes with an agent presenting a complex of a MAGE-A3 HLA-B35 binding peptide comprising SEQ ID NO:56 and a HLA-B35 molecule, in an amount sufficient to selectively enrich the population of T lymphocytes with the T lymphocytes specific for one of the complexes. In certain embodiments, the agent is an antigen presenting cell which expresses a HLA-B35 molecule contacted with a MAGE-A3 protein or a HLA binding fragment thereof which comprises SEQ ID NO:56. In other embodiments, the MAGE-A3 HLA-B35 binding peptide is selected from the group consisting of (i) SEQ ID NO:56, SEQ ID NO:57 and SEQ ID NO:59, and (ii) functional variants of the peptides of (i).

According to a further aspect of the invention, methods for treating a subject, typed as HLA-B35 positive, having a disorder characterized by expression of MAGE-A3 are provided. The methods include administering to the subject an amount of a MAGE-A3 HLA-B35 binding peptide comprising SEQ ID NO:56 sufficient to ameliorate the disorder. In some embodiments, the MAGE-A3 HLA-B35 binding peptide is selected from the group consisting of (i) peptides which consist of a fragment of the amino acid sequence of SEQ ID NO:55, (ii) peptides which comprise the amino acid sequence of SEQ ID NO:59, and (iii) functional variants of the peptides of (i) and (ii). In other embodiments, the methods further include administering to the subject at least one isolated HLA binding peptide selected from the group consisting of (1) MAGE-A3 HLA class I binding peptides other than peptides comprising SEQ ID NO:59, (2) MAGE-A3 HLA class II binding peptides, and (3) HLA class I or class II binding peptide of a non-MAGE-A3 tumor antigen, in an amount sufficient to ameliorate the disorder.

Other methods include administering to the subject an amount of an agent which enriches selectively in the subject the presence of complexes of a HLA-B35 molecule and a MAGE-A3 HLA-B35 binding peptide comprising SEQ ID NO:56, in an amount sufficient to ameliorate the disorder. In some embodiments, the agent includes a MAGE-A3 HLA-B35 binding peptide selected from the group consisting of (i) peptides which consist of a fragment of the amino acid sequence of SEQ ID NO:55, (ii) peptides which comprise the amino acid sequence of SEQ ID NO:59, and (iii) functional variants of the peptides of (i) and (ii).

Still other methods of treatment are provided, that include administering to the subject an amount of autologous T lymphocytes sufficient to ameliorate the disorder, wherein the T lymphocytes are specific for complexes of a HLA-B35 molecule and a MAGE-A3 HLA-B35 binding peptide comprising SEQ ID NO:56. In certain embodiments, the MAGE-A3 HLA-B35 binding peptide is selected from the group consisting of (i) peptides which consist of a fragment of the amino acid sequence of SEQ ID NO:55, (ii) peptides which comprise the amino acid sequence of SEQ ID NO:59, and (iii) functional variants of the peptides of (i) and (ii).

According to another aspect of the invention, methods for identifying functional variants of a MAGE-A3 HLA-B35 binding peptide are provided. The methods include providing a MAGE-A3 HLA-B35 binding peptide comprising SEQ ID NO:56, a HLA-B35 molecule, and a T cell which is stimulated by the MAGE-A3 HLA binding peptide presented by the HLA-B35 molecule. The methods also include mutating a first amino acid residue of the MAGE-A3 HLA-B35 binding peptide to prepare a variant peptide; and determining the binding of the variant peptide to the HLA-B35 molecule or the stimulation of the T cell. Binding of the variant peptide to the HLA binding molecule or stimulation of the T cell by the variant peptide presented by the HLA binding molecule indicates that the variant peptide is a functional variant.

In certain embodiments of the foregoing methods, the MAGE-A3 HLA-B35 binding peptide is selected from the group consisting of (i) peptides which consist of a fragment of the amino acid sequence of SEQ ID NO:55, (ii) peptides which comprise the amino acid sequence of SEQ ID NO:59. In other embodiments, the methods also include comparing the stimulation of the T cell by the MAGE-A3 HLA binding peptide and the stimulation of the T cell by the functional variant as a determination of the effectiveness of the stimulation of the T cell by the functional variant. Isolated functional variants of a MAGE-A3 HLA-B35 binding peptide identified by the foregoing methods also are provided.

In yet another aspect of the invention, expression vectors are provided. The expression vectors include a nucleotide sequence which encodes an amino acid sequence selected from the group consisting of SEQ ID NO:56, SEQ ID NO:57 and SEQ ID NO:59, operably linked to a promoter, and a nucleotide sequence which encodes the amino acid sequence of a HLA-B35 molecule, operably linked to a promoter. The invention also includes host cells transfected or transformed with the foregoing expression vectors.

According to still another aspect of the invention, isolated T lymphocytes are provided which selectively binds a complex of a HLA-B35 molecule and a MAGE-A3 HLA binding peptide which comprises the amino acid sequence of SEQ ID NO:56. In certain embodiments, the MAGE-A3 HLA binding peptide is selected from the group consisting of (i) peptides which consist of a fragment of the amino acid sequence of SEQ ID NO:55, (ii) peptides which comprise the amino acid sequence of SEQ ID NO:59, and (iii) functional variants of the peptides of (i) and (ii).

Also provided are isolated antigen presenting cells which include a complex of a HLA-B35 molecule and a MAGE-A3 HLA binding peptide which comprises the amino acid sequence of SEQ ID NO:56. In certain embodiments, the MAGE-A3 HLA binding peptide is selected from the group consisting of (i) peptides which consist of a fragment of the amino acid sequence of SEQ ID NO:55, (ii) peptides which comprise the amino acid sequence of SEQ ID NO:59, and (iii) functional variants of the peptides of (i) and (ii).

In accordance with another aspect of the invention, vaccine compositions are provided. The vaccine compositions include a cell selected from the foregoing T lymphocytes and antigen presenting cells, and a pharmaceutically acceptable carrier. Preferably the vaccine compositions also include an adjuvant.

According to a further aspect of the invention, methods for identifying a candidate mimetic of a MAGE-A3 HLA-B35 binding peptide are provided. The methods include providing a HLA-B35 molecule, contacting the HLA molecule with a test molecule, and determining the binding of the test molecule to the HLA molecule. A test molecule which binds to the HLA molecule is a candidate mimetic of the MAGE-A3 HLA binding peptide. In certain embodiment, the methods also include contacting the HLA molecule with a HLA-B35 binding molecule comprising the amino acid sequence of SEQ ID NO:56, and determining the binding of the HLA binding molecule to the HLA molecule in the presence and the absence of the test molecule. In other embodiments, the foregoing methods also include forming a complex of the HLA molecule and the candidate mimetic, contacting the complex with a T cell which binds to a complex of a HLA molecule and the MAGE-A3 HLA binding peptide, and assaying activation of the T cell. In certain of these embodiments, activation of the T cell is indicated by a property selected from the group consisting of proliferation of the T cell, interferon-γ production by the T cell, tumor necrosis factor production by the T cell, and cytolysis of a target cell by the T cell. Also provided according to the invention are isolated mimetics of a MAGE-A3 HLA-B35 binding peptide identified by the foregoing methods.

The invention also provides pharmaceutical preparations containing any one or more of the compositions described herein. Such pharmaceutical preparations can include pharmaceutically acceptable diluent carriers or excipients. The use of such compositions in the preparation of medicaments, particularly medicaments for the treatment of cancer also is provided.

Disorders as used herein include cancers, such as melanomas, esophageal carcinomas, lung carcinomas, head and neck carcinomas, breast carcinomas, colorectal carcinomas, myelomas, brain tumors including neuroblastomas, sarcomas, prostate carcinomas, renal carcinomas, bladder carcinomas, hepatocellular carcinomas, seminomas, ovarian tumors, papillary thyroid carcinomas, and gastric carcinomas.

These and other objects of the invention will be described in further detail in connection with the detailed description of the invention.

### BRIEF DESCRIPTION OF THE FIGURES

FIG. 1 depicts the lysis by CTL clone 7 of autologous EBV-B cells infected with vaccinia-MAGE-A1.

FIG. 2 shows that a MAGE-A1 epitope presented by HLA-B35 is recognized by CTL clone 7.

FIG. 3 shows the titration of MAGE-A1 peptides in lysis experiments using CTL clone 7.

FIG. 4 depicts lysis of a HLA-B35 melanoma cell line expressing MAGE-A1.

FIG. 5 depicts the lysis by CTL clone 4.2 of autologous EBV-B cells infected with vaccinia-MAGE-A1.

FIG. 6 shows that a MAGE-A1 epitope presented by HLA-B*4402 is recognized by CTL clone 4.2. FIG. 6A shows recognition by CTLs of COS-7 cells transfected with a MAGE-A1 cDNA and each of the cDNAs coding for putative HLA-presenting molecules.

FIG. 6B shows recognition by CTLs of COS-7 cells transfected with the HLA-B*4402 cDNA and cDNA encoding different MA GE genes.

FIG. 7 shows the MAGE-A1 epitope presented by HLA-B*4402 is encoded by the MAGE-A1 fragment having nucleotides 481-507.

FIG. 8 depicts lysis of a HLA-B*4402 melanoma cell line expressing MAGE-A1.

FIG. 9 shows lysis by LB1841-CTL G6/41 of autologous EBV-B cells infected with vaccinia-MAGE-A3.

FIG. 10 shows that a MAGE-A3 epitope presented by HLA-B35 is recognized by CTL clone 41.

FIG. 11 shows the titration of MAGE-A3 peptides in lysis experiments using CTL clone 41.

FIG. 12 depicts recognition of HLA-B35 melanoma cell lines expressing MAGE-A3: (A) TNF production, (B) specific lysis.

### DETAILED DESCRIPTION OF THE INVENTION

The invention provides isolated MAGE peptides, which are presented by HLA class I molecules, and which stimulate the proliferation and activation of CD8⁺ T lymphocytes. Such peptides are referred to herein as “MAGE immunogenic polypeptides” and “MAGE HLA class I binding peptides” and “MAGE HLA peptides”, peptides specific for MAGE-A1 or MAGE-A3 (e.g., “MAGE-A1 immunogenic polypeptides”, “MAGE-A1 HLA class I binding peptides”, “MAGE-A1 immunogenic polypeptides” and “MAGE-A3 HLA class I binding peptides”) and the like. Hence, one aspect of the invention is the use of these isolated peptides to stimulate the immune system of a subject who is HLA-B35 or HLA-B44 positive to recognize cells that express MAGE-A1 or MAGE-A3, such as tumor cells.

The methodology described herein whereby different viral expression systems are used to identify peptides and their presenting HLA molecule(s) is a sensitive and robust way to identify and select immunologically relevant epitopes of a protein. The examples below show the isolation of peptides which are MAGE-A1 or MAGE-A3 HLA binding peptides. These exemplary peptides are processed translation products of the nucleic acid of SEQ ID NO:1 or SEQ ID NO:54. As such, it will be appreciated by one of ordinary skill in the art that the translation products from which a MAGE immunogenic polypeptide is processed to a final form for presentation may be of any length or sequence so long as they encompass a MAGE HLA binding peptide. In certain instances, the MAGE-A1 HLA binding peptide includes an amino acid sequence as set forth in SEQ ID NOs:5, 8, 9, 10, 12, 14 and 53. MAGE-A3 HLA binding peptides include amino acid sequences as set forth in SEQ ID NOs:56, 57 and 59.

As demonstrated in the examples below, peptides or proteins as small as 8 amino acids are appropriately processed, presented by HLA class I molecules and effective in stimulating CD8⁺ T lymphocytes. The MAGE HLA binding peptides, e.g., SEQ ID NOs:5, 8, 9, 10, 12, 14, 53, 56, 57 and 59, may have one, two, three, four, five, six, seven, eight, nine, ten, or more amino acids added to either or both ends. The added amino acids can correspond to MAGE polypeptides (e.g., SEQ ID NO:2, SEQ ID NO:55), or can be unrelated. As is well known in the art, the antigenic portion of such a peptide is cleaved out under physiological conditions for presentation by an HLA class I molecule.

The MAGE HLA binding peptides of the invention do not include the entire MAGE-A1 or MAGE-A3 polypeptides but do include fragments of the MAGE-A1 or MAGE-A3 polypeptides that include the MAGE HLA binding peptides disclosed herein. Thus, for example, fragments of MAGE-A1 or MAGE-A3 polypeptides that include the HLA binding peptides and are 9, 10, 11, 12, 13, 14, 15, 16, 17, 18, 19, 20, 21, 22, 23, 24, 25, 26, 27, 28, 29, 30 amino acids, and so on up to the entire length of the MAGE-A1 or MAGE-A3 proteins less one amino acid are included in the invention. It is intended that the foregoing set of MAGE protein fragments includes each and every integer from 9 amino acids up to the entire length of the proteins less one amino acid.

As noted above, the invention embraces functional variants of the MAGE HLA binding peptides. As used herein, a “functional variant” or “variant” of a MAGE HLA binding peptide is a molecule which contains one or more modifications to the primary amino acid sequence (including inter-amino acid bonds) of the MAGE HLA binding peptide and retains the HLA class I binding properties disclosed herein, as well as the ability to stimulate proliferation and/or activation of CD8⁺ T lymphocytes. Modifications which create a MAGE immunogenic polypeptide functional variant can be made for example 1) to enhance a property of a MAGE HLA binding peptide, such as peptide stability in an expression system or the stability of protein-protein binding such as HLA-peptide binding; 2) to provide a novel activity or property to a MAGE immunogenic polypeptide, such as addition of an antigenic epitope or addition of a detectable moiety; or 3) to provide a different amino acid sequence that produces the same or similar T cell stimulatory properties.

Preferably, the modifications to the amino acid sequence do not alter residues that are known to be important for the HLA binding activities of the peptides, i.e., the anchor residues that confer HLA binding. One of ordinary skill in the art will know these residues and will preferentially substitute other amino acid residues in the peptides in making variants. It is possible also to use other members of the consensus amino acids for a particular anchor residue. For example, consensus anchor residues for HLA-B35 are P in position 2 and Y, F, M, L or I in position 9. Therefore, if position 9 of a peptide was tyrosine (Y), one could substitute phenylalanine (F), methionine (M), leucine (L) or isoleucine (I) and maintain a consensus amino acid at the anchor residue positions of the peptide.

In preparing variants, it is preferred that at least one of the “anchor residue”amino acids of the peptides as disclosed herein is maintained. Accordingly, for a nine amino acid peptide, as many as eight amino acids can be changed to prepare variant peptides. As most HLA class I molecules bind two anchor residues in HLA binding peptides, it is more preferable that at least the two anchor residues are not changed (more or fewer residues depending on the number of anchor residues for a particular HLA molecule). Thus preferably 7 or fewer amino acids of a peptide will be altered to form variant peptides. Still more preferably, fewer than 7 amino acids (6, 5, 4, 3, 2 or 1) will be altered. For peptides of lengths other than nine amino acids, it is preferred that 1, 2, 3, 4, 5, 6, 7, 8, and so on up to one fewer than the length of the peptide are changed when preparing variant peptides.

It is possible, however, to prepare functional variant peptides that do not retain any of the residues described herein. The three dimensional shape of the peptide may be mimicked by other peptide sequences that bear no sequence relation to the original amino acid sequence. Such functional variant peptides can be identified, for example, by competing for HLA binding with one of the peptides disclosed herein. A set of peptides (randomly or nonrandomly synthesized) can be prepared and tested in standard HLA binding assays for competition with the HLA-MAGE peptide binding activity. Those peptides that reduce the HLA-MAGE peptide binding are considered potential functional variants. The sequence of the peptides can be determined and additional tests performed to confirm the variant peptide's function.

Modifications to a MAGE HLA binding peptide can be made to a nucleic acid which encodes the peptide, and can include deletions, point mutations, truncations, amino acid substitutions and additions of amino acids. Alternatively, modifications can be made directly to the polypeptide, such as by cleavage, addition of a linker molecule, addition of a detectable moiety, such as biotin, addition of a fatty acid, substitution of one amino acid for another and the like. Modifications also embrace fusion proteins comprising all or part of the MAGE immunogenic polypeptide amino acid sequence.

The amino acid sequence of MAGE immunogenic polypeptides may be of natural or non-natural origin, that is, they may comprise a natural MAGE immunogenic polypeptide molecule (e.g., a portion of the MAGE-A1 or MAGE-A3 proteins) or may comprise a modified sequence as long as the amino acid sequence retains the ability to stimulate cytolytic T cells when presented and retains the property of binding to a HLA class I molecule such as a HLA-B35 or a HLA-B44 molecule. For example, MAGE-A1 immunogenic polypeptides in this context may be fusion proteins of a MAGE-A1 HLA binding peptide and unrelated amino acid sequences, synthetic peptides of amino acid sequences shown in SEQ ID NOs:5, 8, 9, 10, 12, 14 and 53, labeled peptides, peptides isolated from patients with a MAGE-A1 expressing cancer, peptides isolated from cultured cells which express MAGE-A1, peptides coupled to nonpeptide molecules (for example in certain drug delivery systems) and other molecules which include the amino acid sequence of SEQ ID NO:10. Likewise, MAGE-A3 immunogenic polypeptides may be based on or include the MAGE-A3 HLA binding peptide sequences disclosed herein, in the form of fusion proteins, synthetic peptides, labeled peptides, peptides isolated from patients with a MAGE-A3 expressing cancer, peptides isolated from cultured cells which express MAGE-A3, peptides coupled to nonpeptide molecules, and the like.

Preferably, MAGE HLA binding peptides are non-hydrolyzable. To provide such peptides, one may select MAGE binding peptides from a library of non-hydrolyzable peptides, such as peptides containing one or more D-amino acids or peptides containing one or more non-hydrolyzable peptide bonds linking amino acids. Alternatively, one can select peptides which are optimal for inducing CD8⁺ T lymphocytes and then modify such peptides as necessary to reduce the potential for hydrolysis by proteases. For example, to determine the susceptibility to proteolytic cleavage, peptides may be labeled and incubated with cell extracts or purified proteases and then isolated to determine which peptide bonds are susceptible to proteolysis, e.g., by sequencing peptides and proteolytic fragments. Alternatively, potentially susceptible peptide bonds can be identified by comparing the amino acid sequence of a MAGE immunogenic polypeptide with the known cleavage site specificity of a panel of proteases. Based on the results of such assays, individual peptide bonds which are susceptible to proteolysis can be replaced with non-hydrolyzable peptide bonds by in vitro synthesis of the peptide.

Many non-hydrolyzable peptide bonds are known in the art, along with procedures for synthesis of peptides containing such bonds. Non-hydrolyzable bonds include -psi[CH₂NH]— reduced amide peptide bonds, -psi[COCH₂]— ketomethylene peptide bonds, -psi[CH(CN)NH]— (cyanomethylene)amino peptide bonds, -psi[CH₂CH(OH)]— hydroxyethylene peptide bonds, -psi[CH₂O]— peptide bonds, and -psi[CH₂S]— thiomethylene peptide bonds.

Nonpeptide analogs of peptides, e.g., those which provide a stabilized structure or lessened biodegradation, are also contemplated. Peptide mimetic analogs can be prepared based on a selected MAGE-A1 HLA binding peptide by replacement of one or more residues by nonpeptide moieties. Preferably, the nonpeptide moieties permit the peptide to retain its natural confirmation, or stabilize a preferred, e.g., bioactive, confirmation. One example of methods for preparation of nonpeptide mimetic analogs from peptides is described in Nachman et al., Regul Pept. 57:359-370 (1995). Peptide mimetics also can be selected from libraries of synthetic compounds (e.g. combinatorial libraries of small organic molecules) or natural molecules according to the HLA binding properties and/or T cell stimulatory properties of such molecule. Assays for identification of mimetics of a MAGE immunogenic polypeptide from libraries such as binding assays are well known in the art. Peptide as used herein embraces all of the foregoing.

If a variant involves a change to an amino acid of a MAGE immunogenic polypeptide (e.g., SEQ ID NOs:5, 8, 9, 10, 12, 14, 53, 56, 57 and 59), functional variants of the MAGE immunogenic polypeptide having conservative amino acid substitutions typically will be preferred, i.e., substitutions which retain a property of the original amino acid such as charge, hydrophobicity, conformation, etc. Examples of conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Other methods for identifying functional variants of the MAGE immunogenic polypeptides are provided in a published PCT application of Strominger and Wucherpfennig (PCT/US96/03182). These methods rely upon the development of amino acid sequence motifs to which potential epitopes may be compared. Each motif describes a finite set of amino acid sequences in which the residues at each (relative) position may be (a) restricted to a single residue, (b) allowed to vary amongst a restricted set of residues, or (c) allowed to vary amongst all possible residues. For example, a motif might specify that the residue at a first position may be any one of the residues valine, leucine, isoleucine, methionine, or phenylalanine; that the residue at the second position must be histidine; that the residue at the third position may be any amino acid residue; that the residue at the fourth position may be any one of the residues valine, leucine, isoleucine, methionine, phenylalanine, tyrosine or tryptophan; and that the residue at the fifth position must be lysine.

Sequence motifs for MAGE HLA binding peptide functional variants can be developed by analysis of the binding domains or binding pockets of major histocompatibility complex HLA-B35 or HLA-B44 proteins and/or the T cell receptor (“TCR”) contact points of the MAGE immunogenic polypeptides disclosed herein. By providing a detailed structural analysis of the residues involved in forming the HLA class I binding pockets, one of ordinary skill in the art is enabled to make predictions of sequence motifs for binding to any of the HLA class I proteins. As shown below, the sequence of the MAGE-A3 HLA-B35 binding peptide is unexpected because it would not have been predicted using these methods, i.e., it does not conform to the consensus HLA-B35 binding motifs. It is possible, however, given the sequence of the MAGE-A3 HLA-B35 binding peptide to modify the peptide to create functional variants, to increase binding to HLA, etc.

Using these sequence motifs as search, evaluation, or design criteria, one of ordinary skill in the art is enabled to identify classes of peptides (functional variants of the MAGE-A1 and MAGE-A3 HLA binding peptides disclosed herein) which have a reasonable likelihood of binding to a particular HLA molecule and of interacting with a T cell receptor to induce T cell response. These peptides can be synthesized and tested for activity as described herein. Use of these motifs, as opposed to pure sequence homology (which excludes many peptides which are antigenically similar but quite distinct in sequence) or sequence homology with unlimited “conservative” substitutions (which admits many peptides which differ at critical highly conserved sites), represents a method by which one of ordinary skill in the art can evaluate peptides for potential application in the treatment of disease.

The Strominger and Wucherpfennig PCT application, and references cited therein, all of which are incorporated by reference, describe the HLA class II and TCR binding pockets which contact residues of a HLA class II peptide. Likewise, by keeping the residues which are likely to bind in the HLA class I and/or TCR binding pockets constant or permitting only specified substitutions, functional variants of the MAGE-A1 HLA binding peptides can be prepared which retain binding to HLA class I and T cell receptor.

Localization of one or more antigenic peptides in a protein sequence can be aided by HLA peptide binding predictions made according to established rules for binding potential (e.g., Parker et al, J. Immunol. 152:163, 1994; Rammensee et al., Immunogenetics 41:178-228, 1995). HLA binding predictions can conveniently be made using algorithms available via the Internet on the National Institutes of Health World Wide Web site (bimas.dcrt.nih.gov) and the HLA site of Prof. Rammensee at the University of Tubingen (http://134.2.96.221/scripts/hlaserver.dll/EpPredict).

Thus methods for identifying functional variants of a MAGE-A1 immunogenic polypeptide are provided. In general, the methods include selecting a MAGE HLA binding peptide (e.g., the MAGE-A1 and MAGE-A3 peptides presented herein), a HLA class I binding molecule which binds the MAGE HLA binding peptide, and a T cell which is stimulated by the MAGE HLA binding peptide presented by the HLA class I binding molecule. In preferred embodiments, the MAGE-A1 immunogenic polypeptide comprises the amino acid sequence of SEQ ID NO:8 or SEQ ID NO:10. More preferably, the peptide comprises or consists of the amino acid sequence of any one of SEQ ID NOs:5, 8, 9, 10, 12, 14 and 53. In other preferred embodiments, the MAGE-A3 immunogenic polypeptide comprises the amino acid sequence of SEQ ID NO:56. More preferably, the peptide comprises or consists of the amino acid sequence of any one of SEQ ID NOs:56, 57 and 59.

As an example of the foregoing method, a first amino acid residue of the MAGE-A1 HLA binding peptide is mutated to prepare a variant peptide. The amino acid residue can be mutated according to the principles of HLA and T cell receptor contact points set forth above. Any method for preparing variant peptides can be employed, such as synthesis of the variant peptide, recombinantly producing the variant peptide using a mutated nucleic acid molecule, and the like. The binding of the variant peptide to HLA class I binding molecule and/or stimulation of the T cell are then determined according to standard procedures. For example, as exemplified below, the variant peptide can be contacted with an antigen presenting cell which contains the HLA class I molecule which binds the MAGE-A1 HLA binding peptide to form a complex of the variant peptide and the antigen presenting cell. This complex can then be contacted with a T cell which recognizes the MAGE-A1 HLA binding peptide presented by the HLA class I binding molecule. T cells can be obtained from a patient having a condition characterized by expression of MAGE-A1. Recognition of variant peptides by the T cells can be determined by measuring an indicator of T cell stimulation such as TNF or IFNγ production.

Binding of the variant peptide to the HLA class I binding molecule and/or stimulation of the T cell by the variant peptide presented by the HLA class I binding molecule indicates that the variant peptide is a functional variant. The methods also can include the step of comparing the stimulation of the T cell by the MAGE-A1 HLA binding peptide and the stimulation of the T cell by the functional variant as a determination of the effectiveness of the stimulation of the T cell by the functional variant. By comparing the functional variant with the MAGE-A1 HLA binding peptide, peptides with substantially equivalent or increased T cell stimulatory properties can be prepared. MAGE-A3 variant peptides can be identified by similar procedures.

Variants of the MAGE binding peptides prepared by any of the foregoing methods can be sequenced, if necessary, to determine the amino acid sequence and thus deduce the nucleotide sequence which encodes such variants.

Thus those nucleic acid sequences which code for a MAGE HLA binding peptide or variant thereof, including allelic variants, are also a part of the invention. In screening for nucleic acids which encode a MAGE immunogenic polypeptide, a nucleic acid hybridization such as a Southern blot or a Northern blot may be performed under stringent conditions, together with a labeled detectable probe, for example, a radioactively labeled probe (e.g., ³²P) or a nonradioactively labeled probe (e.g., chemiluminescent probe prepared using the ECL (enhanced chemiluminescence) direct labeling system of Amersham). The term “stringent conditions” as used herein refers to parameters with which the art is familiar. Nucleic acid hybridization parameters may be found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary stringent conditions include hybridization at 50-65° C. in hybridization buffer (3.5×SSC, 0.02% Ficoll, 0.02% Polyvinyl pyrolidone, 0.02% Bovine Serum Albumin, 25 mM NaH₂PO₄ (pH7), 0.5% SDS, 2mM EDTA). SSC is 0.15 M Sodium Chloride/0.015M Sodium Citrate, pH 7; SDS is Sodium Dodecyl Sulphate; and EDTA is Ethylene diaminetetraacetic acid. After hybridization, the membrane upon which the DNA is transferred can be washed, for example, at 2×SSC at room temperature and then at 0.1 -0.5×SSC/0.1×SDS at temperatures of 42-68° C. After washing the membrane to which DNA encoding a MAGE-A1 immunogenic polypeptide was finally transferred, the membrane can be placed against X-ray film to detect the radioactive signal.

There are other conditions, reagents, and so forth which can used, which result in a similar degree of stringency. The skilled artisan will be familiar with such conditions, and thus they are not given here. It will be understood, however, that the skilled artisan will be able to manipulate the conditions in a manner to permit the clear identification of homologs and alleles of nucleic acids encoding the MAGE immunogenic polypeptides of the invention. The skilled artisan also is familiar with the methodology for screening cells and libraries for expression of such molecules which then are routinely isolated, followed by isolation of the pertinent nucleic acid molecule and sequencing.

The invention also includes the use of nucleic acid sequences which include alternative codons that encode the same amino acid residues of the MAGE immunogenic polypeptides. For example, as disclosed herein, the peptide EADPTGHSYVLV (SEQ ID NO:5) is a MAGE-A1 HLA binding peptide. The leucine residue, for example, can be encoded by the codons CUA, CUC, CUG, CUU, UUA and UUG. Each of the six codons is equivalent for the purposes of encoding a leucine residue. Thus, it will be apparent to one of ordinary skill in the art that any of the leucine-encoding nucleotide triplets may be employed to direct the protein synthesis apparatus, in vitro or in vivo, to incorporate a leucine residue. Similarly, nucleotide sequence triplets which encode other amino acid residues comprising the MAGE-A1 HLA binding peptide of SEQ ID NO:5 include: GUA, GUC, GUG and GUU (valine codons); GGU, GGA, GGG, GGC (glycine codons); UAC and UAU (tyrosine codons). Other amino acid residues may be encoded similarly by multiple nucleotide sequences. Thus, the invention embraces degenerate nucleic acids that differ from the native MAGE immunogenic polypeptide encoding nucleic acids in codon sequence due to the degeneracy of the genetic code.

Preferred nucleic acids encoding MAGE polypeptides are those which preferentially express MAGE immunogenic polypeptides, such as the HLA binding peptide described herein. The MAGE nucleic acids of the invention do not encode the entire MAGE polypeptide but do include nucleotide sequences encoding the MAGE HLA binding peptide.

The invention also provides modified nucleic acid molecules which include additions, substitutions and deletions of one or more nucleotides. In preferred embodiments, these modified nucleic acid molecules and/or the polypeptides they encode retain at least one activity or function of the unmodified nucleic acid molecule and/or the polypeptides, such as antigenicity, enzymatic activity, receptor binding, formation of complexes by binding of peptides by MHC class I and class II molecules, etc. In certain embodiments, the modified nucleic acid molecules encode modified polypeptides, preferably polypeptides having conservative amino acid substitutions as are described elsewhere herein. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under stringent conditions known to one of skill in the art.

For example, modified nucleic acid molecules which encode polypeptides having single amino acid changes can be prepared (e.g., preferably not those amino acids which are contact points for HLA binding). Each of these nucleic acid molecules can have one, two or three nucleotide substitutions exclusive of nucleotide changes corresponding to the degeneracy of the genetic code as described herein. Likewise, modified nucleic acid molecules which encode polypeptides having two amino acid changes can be prepared which have, e.g., 2-6 nucleotide changes. Numerous modified nucleic acid molecules like these will be readily envisioned by one of skill in the art, including for example, substitutions of nucleotides in codons encoding amino acids 2 and 3, 2 and 4, 2 and 5, 2 and 6, and so on. In the foregoing example, each combination of two amino acids is included in the set of modified nucleic acid molecules, as well as all nucleotide substitutions which code for the amino acid substitutions. Additional nucleic acid molecules that encode polypeptides having additional substitutions (i.e., 3 or more), additions or deletions (e.g., by introduction of a stop codon or a splice site(s)) also can be prepared and are embraced by the invention as readily envisioned by one of ordinary skill in the art. Any of the foregoing nucleic acids or polypeptides can be tested by routine experimentation for retention of structural relation or activity to the nucleic acids and/or polypeptides disclosed herein.

It will also be understood that the invention embraces the use of the sequences in expression vectors, as well as to transfect host cells and cell lines, be these prokaryotic (e.g., E. coli), or eukaryotic (e.g., dendritic cells, CHO cells, COS cells, yeast expression systems and recombinant baculovirus expression in insect cells). The expression vectors require that the pertinent sequence, i.e., those described herein, be operably linked to a promoter. In addition, as it has been found that human HLA-B35 and HLA-B44 molecules present MAGE-A1 HLA class I binding peptides, and that HLA-B35 molecules present MAGE-A3 HLA class I binding peptides, the expression vector may also include a nucleic acid sequence coding for a HLA-B35 or a HLA-B44 molecule. (For other class I or class II binding peptides, different HLA molecules can be used.) In a situation where the vector contains both coding sequences, it can be used to transfect a cell which does not normally express either one. The MAGE HLA class I binding peptide coding sequence may be used alone, when, e.g. the host cell already expresses a HLA-B35 or a HLA-B44 molecule. Of course, there is no limit on the particular host cell which can be used as the vectors which contain the two coding sequences may be used in host cells which do not express HLA-B35 or a HLA-B44 molecules if desired, and the nucleic acid coding for the MAGE HLA class I binding peptide can be used in antigen presenting cells which express an appropriate HLA-B35 or a HLA-B44 molecule. As used herein, “a HLA-B35 molecule” includes the subtypes HLA-B*35011, B*35012, B*3502, B*3503, B*3504, B*3505, B*3506, B*3507, B*3508, B*35091, B*35092, B*3510, B*3511, B*3512, B*3513, B*3514, B*3515, B*3516, B*3517, B*3518, B*3519, B*3520, B*3521, B*3522, B*3523, B*3524, B*3525, B*3526, B*3527, B*3528, B*3529, B*3530, B*3531, B*3532, B*3533, B*3534, B*3535, B*3536, B*3537. As used herein, “a HLA-B44 molecule” includes the subtypes HLA-B*4401, B*44021, B*44022, B*44031, B*44032, B*4404, B*4405, B*4406, B*4407, B*4408, B*4409, B*4410, B*4411, B*4412, B*4413, B*4414, B*4415, B*4416, B*4417, B*4418, B*4419N, B*4420, B*4421, B*4422, B*4423N, B*4424. HLA-B35 and -B44 molecules also include the subtypes which can be found in Bodmer et al., Tissue Antigens 49:297, 1996. A listing of presently identified HLA-B35 and -B44 subtypes can be found on the IMGT/HLA database at internet URL http://www.ebi.ac.uk/imgt/hla/.

It will also be understood that the invention embraces the use of the sequences in expression vectors including recombinant plasmids, phagemids, viruses and the like, as well as to transfect host cells and cell lines, be these prokaryotic (e.g., E. coli), or eukaryotic (e.g., dendritic cells, CHO cells, COS cells, yeast expression systems and recombinant baculovirus expression in insect cells). The expression vectors require that the pertinent sequence, i.e., those described herein, be operably linked to a promoter. Delivery of expression vectors containing the MAGE sequences in vivo and/or in vitro can be via the use of nucleic acid delivery systems known in the art (see, e.g., Allsopp et al., Eur. J. Immunol 26(8):1951-1959, 1996). Recombinant vectors including viruses selected from the group consisting of adenoviruses, adeno-associated viruses, poxviruses including vaccinia viruses and attenuated poxviruses such as ALVAC, NYVAC, Semliki Forest virus, Venezuelan equine encephalitis virus, retroviruses, Sindbis virus, Ty virus-like particle, plasmids (e.g. “naked” DNA), bacteria (e.g. the bacterium Bacille Calmette Guerin, attenuated Salmonella), and the like can be used in such delivery, for example, for use as a vaccine. Other viruses, expression vectors and the like which are useful in preparation of a vaccine are known to one of ordinary skill in the art. One can test the MAGE delivery systems in standard model systems such as mice to determine efficacy of the delivery system. The systems also can be tested in human clinical trials.

In addition, non-MAGE-A1 and non-MAGE-A3 tumor associated peptides also can be administered to increase immune response via HLA class I and/or class II. It is well established that cancer cells can express more that one tumor associated gene. It is within the scope of routine experimentation for one of ordinary skill in the art to determine whether a particular subject expresses additional tumor associated genes, and then include HLA class I and/or HLA class II binding peptides derived from expression products of such genes in MAGE compositions and vaccines.

Especially preferred are nucleic acids encoding a series of epitopes, known as “polytopes”. The epitopes can be arranged in sequential or overlapping fashion (see, e.g., Thomson et al., Proc. Natl. Acad. Sci. USA 92:5845-5849, 1995; Gilbert et al., Nature Biotechnol. 15:1280-1284, 1997), with or without the natural flanking sequences, and can be separated by unrelated linker sequences if desired. The polytope is processed to generated individual epitopes which are recognized by the immune system for generation of immune responses.

Thus, for example, MAGE-A1 HLA binding peptides such as SEQ ID NOs:5, 8, 9, 10, 12, 14 and 53, and/or MAGE-A3 HLA binding peptides such as SEQ ID NOs:56, 57 and 59, each of which are presented by MHC molecules and recognized by CTLs (or T helper lymphocytes) can be combined with peptides from other tumor rejection antigens (e.g. by preparation of hybrid nucleic acids or polypeptides) to form “polytopes”. Exemplary tumor associated peptide antigens that can be administered to induce or enhance an immune response are derived from tumor associated genes and encoded proteins including MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-1, GAGE-2, GAGE-3, GAGE-4, GAGE-5, GAGE-6, GAGE-7, GAGE-8, GAGE-9, BAGE-1, RAGE-1, LB33/MUM-1, PRAME, NAG, MAGE-Xp2 (MAGE-B2), MAGE-Xp3 (MAGE-B3), MAGE-Xp4 (MAGE-B4), tyrosinase, brain glycogen phosphorylase, Melan-A, MAGE-C1, MAGE-C2, MAGE-C3, MAGE-C4, MAGE-C5, NY-ESO-1, LAGE-1, SSX-1, SSX-2 (HOM-MEL-40), SSX-1, SSX-4, SSX-5, SCP-1 and CT-7. For example, antigenic peptides characteristic of tumors include those listed in Table I below.

TABLE I
Exemplary Antigens

| | | | | SEQ ID |
|---|---|---|---|---|
| Gene | MHC | Peptide | Position | NO: |
| MAGE- | HLA-A1 | EADPTGHSY | 161-169 | 8 |
| A1 | HLA- | SAYGEPRKL | 230-238 | 17 |
| | Cw16 | | | |
| MAGE- | HLA-A1 | EVDPIGHLY | 168-176 | 18 |
| A3 | HLA-A2 | FLWGPRALV | 271-279 | 19 |
| | HLA-B44 | MEVDPIGHLY | 167-176 | 20 |
| BAGE | HLA- | AARAVFLAL | 2-10 | 21 |
| | Cw16 | | | |
| GAGE-1,2 | HLA- | YRPRPRRY | 9-16 | 22 |
| | Cw16 | | | |
| RAGE | HLA-B7 | SPSSNRIRNT | 11-20 | 23 |
| GnT-V | HLA-A2 | VLPDVFIRC(V) | 2-10/11 | 24,25 |
| MUM-1 | HLA-B44 | EEKLIVVLF | exon 2/ | 26 |
| | | EEKLSVVLF | intron | 27 |
| | | (wild type) | | |
| CDK4 | HLA-A2 | ACDPHSGHFV | 23-32 | 28 |
| | | ARDPHSGHFV | | 29 |
| | | (wild type) | | |
| β-catenin | HLA-A24 | SYLDSGIHF | 29-37 | 30 |
| | | SYLDSGIHS | | 31 |
| | | (wild type) | | |
| Tyrosinase | HLA-A2 | MLLAVLYCL | 1-9 | 32 |
| | HLA-A2 | YMNGTMSQV | 369-377 | 33 |
| | HLA-A2 | YMDGTMSQV | 369-377 | 49 |
| | HLA-A24 | AFLPWHRLF | 206-214 | 34 |
| | HLA-B44 | SEIWRDIDF | 192-200 | 35 |
| | HLA-B44 | YEIWRDIDF | 192-200 | 36 |
| | HLA-DR4 | QNILLSNAPLGPQFP | 56-70 | 37 |
| | HLA-DR4 | DYSYLQDSDPDSFQD | 448-462 | 38 |
| Melan- | HLA-A2 | (E)AAGIGILTV | 26/27-35 | 39, 40 |
| A^{MART-1} | HLA-A2 | ILTVILGVL | 32-40 | 41 |
| gp100 | HLA-A2 | KTWGQYWQV | 154-162 | 42 |
| ^{Pmel117} | HLA-A2 | ITDQVPFSV | 209-217 | 43 |
| | HLA-A2 | YLEPGPVTA | 280-288 | 44 |
| | HLA-A2 | LLDGTATLRL | 457-466 | 45 |
| | HLA-A2 | VLYRYGSFSV | 476-485 | 46 |
| PRAME | HLA- | LYVDSLFFL | 301-309 | 47 |
| | A24 | | | |
| MAGE- | HLA- | KISGGPRISYPL | 292-303 | 48 |
| A6 | Cw16 | | | |
| NY- | HLA-A2 | SLLMWITQCFL | 157-167 | 50 |
| ESO-1 | HLA-A2 | SLLMWITQC | 157-165 | 51 |
| | HLA-A2 | QLSLLMWIT | 155-163 | 52 |

Other examples of HLA class I and HLA class II binding peptides will be known to one of ordinary skill in the art. For example, see the following references: Coulie, Stem Cells 13:393-403, 1995; Traversari et al., J. Exp. Med. 176:1453-1457, 1992; Chaux et al., J. Immunol 163:2928-2936, 1999; Fujie et al., Int. J. Cancer 80:169-172, 1999; Tanzarella et al., Cancer Res. 59:2668-2674, 1999; van der Bruggen et al., Eur. J. Immunol. 24:2134-2140, 1994; Chaux et al., J. Exp. Med. 189:767-778, 1999; Kawashima et al, Hum. Immunol. 59:1-14, 1998; Tahara et al., Clin. Cancer Res. 5:2236-2241, 1999; Gaugler et al., J. Exp. Med. 179:921-930, 1994; van der Bruggen et al., Eur. J. Immunol. 24:3038-3043, 1994; Tanaka et al., Cancer Res. 57:4465-4468, 1997; Oiso et al., Int. J. Cancer 81:387-394, 1999; Herman et al., Immunogenetics 43:377-383, 1996; Manici et al., J. Exp. Med. 189:871-876, 1999; Duffour et al., Eur. J Immunol. 29:3329-3337, 1999; Zorn et al., Eur. J. Immunol. 29:602-607, 1999; Huang et al., J. Immunol. 162:6849-6854, 1999; Boël et al., Immunity 2:167-175, 1995; Van den Eynde et al., J. Exp. Med. 182:689-698, 1995; De Backer et al., Cancer Res. 59:3157-3165, 1999; Jäger et al., J. Exp. Med. 187:265-270, 1998; Wang et al., J. Immunol. 161:3596-3606, 1998; Aarnoudse et al., Int. J. Cancer 82:442-448, 1999; Guilloux et al., J. Exp. Med. 183:1173-1183, 1996; Lupetti et al., J. Exp. Med. 188:1005-1016, 1998; Wölfel et al., Eur. J. Immunol. 24:759-764, 1994; Skipper et al., J. Exp. Med. 183:527-534, 1996; Kang et al., J. Immunol. 155:1343-1348, 1995; Morel et al., Int. J. Cancer 83:755-759, 1999; Brichard et al., Eur. J. Immunol. 26:224-230, 1996; Kittlesen et al., J. Immunol. 160:2099-2106, 1998; Kawakami et al., J. Immunol. 161:6985-6992, 1998; Topalian et al., J. Exp. Med. 183:1965-1971, 1996; Kobayashi et al., Cancer Research 58:296-301, 1998; Kawakami et al., J. Immunol. 154:3961-3968, 1995; Tsai et al., J. Immunol. 158:1796-1802, 1997; Cox et al., Science 264:716-719, 1994; Kawakami et al., Proc. Natl. Acad. Sci. USA 91:6458-6462, 1994; Skipper et al., J. Immunol. 157:5027-5033, 1996; Robbins et al., J. Immunol. 159:303-308, 1997; Castelli et al, J. Immunol. 162:1739-1748, 1999; Kawakami et al., J. Exp. Med. 180:347-352, 1994; Castelli et al., J. Exp. Med. 181:363-368, 1995; Schneider et al., Int. J. Cancer 75:451-458, 1998; Wang et al., J. Exp. Med. 183:1131-1140,1996; Wang et al., J. Exp. Med. 184:2207-2216, 1996; Parkhurst et al., Cancer Research 58:4895-4901, 1998; Tsang et al., J. Natl Cancer Inst 87:982-990, 1995; Correale et al., J. Natl Cancer Inst 89:293-300, 1997; Coulie et al., Proc. Natl. Acad. Sci. USA 92:7976-7980, 1995; Wölfel et al., Science 269:1281-1284, 1995; Robbins et al., J. Exp. Med. 183:1185-1192, 1996; Brändle et al., J. Exp. Med. 183:2501-2508, 1996; ten Bosch et al., Blood 88:3522-3527, 1996; Mandruzzato et al., J. Exp. Med. 186:785-793, 1997; Guéguen et al., J. Immunol. 160:6188-6194, 1998; Gjertsen et al., Int. J. Cancer 72:784-790, 1997; Gaudin et al., J. Immunol. 162:1730-1738, 1999; Chiari et al., Cancer Res. 59:5785-5792, 1999; Hogan et al., Cancer Res. 58:5144-5150, 1998; Pieper et al., J. Exp. Med. 189:757-765, 1999; Wang et al., Science 284:1351-1354, 1999; Fisk et al., J. Exp. Med. 181:2109-2117, 1995; Brossart et al., Cancer Res. 58:732-736, 1998; Röpke et al., Proc. Natl. Acad. Sci. USA 93:14704-14707, 1996; Ikeda et al., Immunity 6:199-208, 1997; Ronsin et al., J. Immunol. 163:483-490, 1999; Vonderheide et al., Immunity 10:673-679,1999.

Such HLA class I and HLA class II binding peptides can be used in the invention in a like manner as those disclosed herein. One of ordinary skill in the art can prepare polypeptides comprising one or more MAGE-A1 and/or MAGE-A3 peptides and one or more of the foregoing tumor rejection peptides, or nucleic acids encoding such polypeptides, according to standard procedures of molecular biology.

Thus polytopes are groups of two or more potentially immunogenic or immune response stimulating peptides which can be joined together in various arrangements (e.g. concatenated, overlapping). The polytope (or nucleic acid encoding the polytope) can be administered in a standard immunization protocol, e.g. to animals, to test the effectiveness of the polytope in stimulating, enhancing and/or provoking an immune response.

The peptides can be joined together directly or via the use of flanking sequences to form polytopes, and the use of polytopes as vaccines is well known in the art (see, e.g., Thomson et al., Proc. Acad. Natl. Acad. Sci USA 92(13):5845-5849, 1995; Gilbert et al., Nature Biotechnol. 15(12):1280-1284, 1997; Thomson et al., J. Immunol. 157(2):822-826, 1996; Tam et al., J. Exp. Med. 171(1):299-306, 1990). For example, Tam showed that polytopes consisting of both MHC class I and class II binding epitopes successfully generated antibody and protective immunity in a mouse model. Tam also demonstrated that polytopes comprising “strings” of epitopes are processed to yield individual epitopes which are presented by MHC molecules and recognized by CTLs. Thus polytopes containing various numbers and combinations of epitopes can be prepared and tested for recognition by CTLs and for efficacy in increasing an immune response.

It is known that tumors express a set of tumor antigens, of which only certain subsets may be expressed in the tumor of any given patient. Polytopes can be prepared which correspond to the different combination of epitopes representing the subset of tumor rejection antigens expressed in a particular patient. Polytopes also can be prepared to reflect a broader spectrum of tumor rejection antigens known to be expressed by a tumor type. Polytopes can be introduced to a patient in need of such treatment as polypeptide structures, or via the use of nucleic acid delivery systems known in the art (see, e.g., Allsopp et al., Eur. J. Immunol 26(8):1951-1959, 1996). Adenovirus, pox virus, Ty-virus like particles, adeno-associated virus, plasmids, bacteria, liposomes, etc. can be used in such delivery. One can test the polytope delivery systems in mouse models to determine efficacy of the delivery system. The systems also can be tested in human clinical trials.

As it has been found that human HLA-B35 or HLA-B44 molecules present MAGE immunogenic polypeptides, the expression vector may also include a nucleic acid sequence coding for a HLA-B35 or a HLA-B44 molecule. Nucleic acids encoding single chain soluble HLA/peptide complex including a MAGE-A1 or a MAGE-A3 immunogenic polypeptide fused to a HLA-B35 or a HLA-B44 molecule can be prepared as described by Lone et al. (J. Immunother. 21:283-294, 1998).

In a situation where the vector contains both coding sequences, it can be used to transfect a cell which does not normally express either one. The MAGE-A1 and/or MAGE-A3 immunogenic polypeptide coding sequences may be used alone, when, e.g. the host cell already expresses a HLA-B35 or a HLA-B44 molecule. Of course, there is no limit on the particular host cell which can be used as the vectors which contain the two coding sequences may be used in host cells which do not express HLA-B35 or HLA-B44 molecules if desired, and the nucleic acids coding for MAGE immunogenic polypeptides (such as those described herein for MAGE-A1 and/or MAGE-A3) can be used in antigen presenting cells which express a HLA-B35 or a HLA-B44 molecule.

As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids, phagemids, bacteria and virus genomes as disclosed herein, such as adenovirus, poxvirus and BCG. A cloning vector is one which is able to replicate in a host cell or be replicated after its integration into the genome of a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., P-galactosidase, luciferase or alkaline phosphatase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide. As noted above, certain preferred nucleic acids express only fragments of MAGE-A1 or MAGE-A3 polypeptides which include the HLA binding peptides described herein.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Preferably, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA) encoding a MAGE immunogenic polypeptide, e.g., those described herein that are derived from MAGE-A1 or MAGE-A3. That heterologous DNA (RNA) is placed under operable control of transcriptional elements to permit the expression of the heterologous DNA in the host cell.

Preferred systems for mRNA expression in mammalian cells are those such as pcDNA3.1 (available from Invitrogen, Carlsbad, Calif.) that contain a selectable marker such as a gene that confers G418 resistance (which facilitates the selection of stably transfected cell lines) and the human cytomegalovirus (CMV) enhancer-promoter sequences. Additionally, suitable for expression in primate or canine cell lines is the pCEP4 vector (Invitrogen), which contains an Epstein Barr virus (EBV) origin of replication, facilitating the maintenance of plasmid as a multicopy extrachromosomal element. Another expression vector is the pEF-BOS plasmid containing the promoter of polypeptide Elongation Factor 1α, which stimulates efficiently transcription in vitro. The plasmid is described by Mishizuma and Nagata (Nuc. Acids Res. 18:5322, 1990), and its use in transfection experiments is disclosed by, for example, Demoulin (Mol. Cell. Biol. 16:4710-4716, 1996). Still another preferred expression vector is an adenovirus, described by Stratford-Perricaudet, which is defective for El and E3 proteins (J. Clin. Invest. 90:626-630, 1992). The use of the adenovirus to express proteins for immunization is disclosed by Warnier et al., in intradermal injection in mice for immunization against P1A (Int. J. Cancer, 67:303-310, 1996).

The invention also embraces so-called expression kits, which allow the artisan to prepare a desired expression vector or vectors. Such expression kits include at least separate portions of at least two of the previously discussed materials. Other components may be added, as desired.

The invention as described herein has a number of uses, some of which are described herein. First, the invention permits the artisan to diagnose a disorder characterized by expression of a MAGE immunogenic polypeptide. These methods involve determining expression of, e.g., a MAGE-A1 or MAGE-A3 HLA binding peptide, or a complex of a MAGE-A1 or MAGE-A3 HLA binding peptide and a HLA class I molecule (e.g., HLA-B35 or HLA-B44) in a biological sample. The expression of a peptide or complex of peptide and HLA class I molecule can be determined by assaying with a binding partner for the peptide or complex, such as an antibody. The expression of MAGE-A1 or MAGE-A3 in a biological sample, such as a tumor biopsy, particularly tumors in patients expressing HLA-B35 or HLA-B44, can also be tested by standard PCR amplification protocols using MAGE-A1 or MAGE-A3 primers. An example of tumor expression is presented herein and further disclosure of exemplary conditions and primers for MAGE-A1 amplification can be found in U.S. application Ser. No. 09/018,422.

The diagnostic methods also may involve typing a subject for the HLA molecules that the subject expresses. This can be done using a variety of standard methodologies for screening a biological sample isolated from a subject, including those which are based on the known sequence of HLA molecules, e.g., PCR analysis of HLA type, such as sequence-specific primed polymerase chain reaction (PCR-SSP) or using fluorogenic probes (TaqMan®, PE Biosystems, Foster City, Calif.), hybridization with HLA-specific probes, etc. Other methods include those based on immunological methods (e.g., antibody recognition of HLA).

Preferably, the diagnostic methods involve contacting a biological sample isolated from a subject with an agent specific for the MAGE-A1 or MAGE-A3 HLA binding peptides to detect the presence of the MAGE-A1 or MAGE-A3 HLA binding peptides in the biological sample. As used herein, “contacting” means placing the biological sample in sufficient proximity to the agent and under the appropriate conditions of, e.g., concentration, temperature, time, and ionic strength, to allow the specific interaction between the agent and MAGE-A1 or MAGE-A3 HLA binding peptides that are present in the biological sample. In general, the conditions for contacting the agent with the biological sample are conditions known by those of ordinary skill in the art to facilitate a specific interaction between a molecule and its cognate (e.g., a protein and its receptor cognate, an antibody and its protein antigen cognate, a nucleic acid and its complementary sequence cognate) in a biological sample. Exemplary conditions for facilitating a specific interaction between a molecule and its cognate are described in U.S. Pat. No. 5,108,921, issued to Low et al.

The biological sample can be located in vivo or in vitro. For example, the biological sample can be a tissue in vivo and the agent specific for the MAGE-A1 or MAGE-A3 immunogenic polypeptides can be used to detect the presence of such molecules in the tissue. Alternatively, the biological sample can be located in vitro (e.g., a blood sample, tumor biopsy, tissue extract). In a particularly preferred embodiment, the biological sample can be a cell-containing sample, more preferably a sample containing tumor cells.

The invention also permits the artisan to treat a subject having a disorder characterized by expression of a MAGE-A1 and/or MAGE-A3 immunogenic polypeptide. Treatments include administering an agent which enriches in the subject a complex of a MAGE-A1 and/or MAGE-A3 HLA binding peptide and a HLA class I molecule, and administering CD8⁺ T lymphocytes which are specific for such complexes. Agents useful in the foregoing treatments include MAGE-A1 and/or MAGE-A3 immunogenic polypeptides (e.g., HLA binding peptides) and functional variants thereof, complexes of such peptides and HLA class I binding molecules (e.g. HLA-B35 or HLA-B44), antigen presenting cells bearing complexes of a MAGE-A1 and/or a MAGE-A3 immunogenic polypeptide and one or more HLA class I binding molecules, soluble single chain fusions of HLA and MAGE-A1 or MAGE-A3 polypeptides, and the like. The invention also permits an artisan to selectively enrich a population of T lymphocytes for CD8⁺ T lymphocytes specific for a MAGE-A1 and/or a MAGE-A3 HLA binding peptide.

The isolation of the MAGE-A1 and MAGE-A3 HLA binding peptides also makes it possible to isolate or design nucleic acids which encode the MAGE-A1 and/or MAGE-A3 HLA binding peptides. Nucleic acids can be used to produce the MAGE-A1 and/or MAGE-A3 HLA binding peptides or proteins containing such peptides in vitro or in vivo in prokaryotic or eukaryotic host cells. A variety of methodologies well-known to the skilled practitioner can be utilized to obtain isolated MAGE-A1 and/or MAGE-A3 HLA binding peptides. For example, an expression vector may be introduced into cells to cause production of the peptides. In another method, mRNA transcripts may be microinjected or otherwise introduced into cells to cause production of the encoded peptides. Translation of mRNA in cell-free extracts such as the reticulocyte lysate system also may be used to produce peptides. Peptides comprising the MAGE-A1 and/or MAGE-A3 HLA binding peptides of the invention may also be synthesized in vitro. Those skilled in the art also can readily follow known methods for isolating peptides in order to obtain isolated MAGE-A1 and/or MAGE-A3 HLA binding peptides. These include, but are not limited to, immunochromotography, HPLC, size-exclusion chromatography, ion-exchange chromatography and immune-affinity chromatography.

These isolated MAGE-A1 and/or MAGE-A3 HLA binding peptides, or complexes of the peptides and HLA class I molecules, such as a HLA-B35 or a HLA-B44 molecule, may be combined with materials such as adjuvants to produce vaccines useful in treating disorders characterized by expression of the MAGE-A1 and/or MAGE-A3 immunogenic polypeptide. In addition, vaccines can be prepared from cells which present the MAGE-A1 and/or MAGE-A3 HLA binding peptide/HLA complexes on their surface, such as transfected dendritic cells, transfected B cells, non-proliferative transfectants, et cetera. In all cases where cells are used as a vaccine, these can be cells transfected with coding sequences for one or both of the components necessary to stimulate CD8⁺ lymphocytes, or be cells which already express both molecules without the need for transfection. Vaccines also encompass expression vectors and naked DNA or RNA, encoding a MAGE-A1 and/or MAGE-A3 HLA binding peptide, precursors thereof, or fusion proteins thereof, which may be produced in vitro and administered via injection, particle bombardment, nasal aspiration and other methods. Vaccines of the “naked nucleic acid” type have been demonstrated to provoke an immunological response including generation of CTLs specific for the peptide encoded by the naked nucleic acid (Science 259:1745-1748, 1993).

The MAGE-A1 and/or MAGE-A3 HLA binding peptides, as well as complexes of MAGE-A1 and/or MAGE-A3 HLA binding peptides and HLA molecules, also may be used to produce antibodies, using standard techniques well known to the art. Standard reference works setting forth the general principles of antibody production include Catty, D., Antibodies, A Practical Approach, Vol. 1, IRL Press, Washington D.C. (1988); Klein, J., Immunology: The Science of Cell-Non-Cell Discrimination, John Wiley and Sons, New York (1982); Kennett, R., et al., Monoclonal Antibodies, Hybridoma, A New Dimension In Biological Analyses, Plenum Press, New York (1980); Campbell, A., Monoclonal Antibody Technology, in Laboratory Techniques and Biochemistry and Molecular Biology, Vol. 13 (Burdon, R. et al. EDS.), Elsevier Amsterdam (1984); and Eisen, H. N., Microbiology, third edition, Davis, B. D. et al. EDS. (Harper & Rowe, Philadelphia (1980).

The antibodies of the present invention thus are prepared by any of a variety of methods, including administering protein, fragments of protein, cells expressing the protein or fragments thereof and an appropriate HLA class I molecule, and the like to an animal to induce polyclonal antibodies. The production of monoclonal antibodies is according to techniques well known in the art.

Significantly, as is well-known in the art, only a small portion of an antibody molecule, the paratope, is involved in the binding of the antibody to its epitope (see, in general, Clark, W. R. (1986) The Experimental Foundations of Modem Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762 and 5,859,205.

Thus, for example, PCT International Publication Number WO 92/04381 teaches the production and use of humanized murine RSV antibodies in which at least a portion of the murine FR regions have been replaced by FR regions of human origin. Such antibodies, including fragments of intact antibodies with antigen-binding ability, are often referred to as “chimeric” antibodies.

Fully human monoclonal antibodies also can be prepared, for example, by immunization of non-human animals transgenic for human immunoglobulin genes. See, for example, U.S. Pat. Nos. 5,814,318, 5,877,397, 6,091,001, 6,114,598.

Thus, as will be apparent to one of ordinary skill in the art, the present invention also provides for F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

Such antibodies may be used for example to identify tissues expressing protein or to purify protein. Antibodies also may be coupled to specific labeling agents for imaging or to antitumor agents, including, but not limited to, methotrexate, radioiodinated compounds, toxins such as ricin, other cytostatic or cytolytic drugs, and so forth. Antibodies prepared according to the invention also preferably are specific for the peptide/HLA complexes described herein.

When “disorder” or “condition” is used herein, it refers to any pathological condition where the MAGE-A1 and/or the MAGE-A3 immunogenic polypeptides are expressed. Such disorders include cancers, including melanomas, esophageal carcinomas, lung carcinomas, head and neck carcinomas, breast carcinomas, colorectal carcinomas, myelomas, brain tumors including neuroblastomas, sarcomas, prostate carcinomas, renal carcinomas, bladder carcinomas, hepatocellular carcinomas, seminomas, ovarian tumors, papillary thyroid carcinomas, and gastric carcinomas.

Some therapeutic approaches based upon the disclosure are premised on inducing a response by a subject's immune system to MAGE-A1 and/or MAGE-A3 immunogenic polypeptide presenting cells. One such approach is the administration of autologous CD8⁺ T cells specific for the complex of a MAGE-A1 HLA binding peptide and/or MAGE-A3 HLA binding peptide and a HLA class I molecule to a subject with abnormal cells of the phenotype at issue. It is within the skill of the artisan to develop such CD8⁺ T cells in vitro. Generally, a sample of cells taken from a subject, such as blood cells, are contacted with a cell presenting the complex and capable of provoking CD8⁺ T lymphocytes to proliferate. The target cell can be a transfectant, such as a transfected COS cell, or a transfected antigen presenting cell bearing HLA class I molecules, such as dendritic cells or B cells. These transfectants present the desired complex of their surface and, when combined with a CD8⁺ T lymphocyte of interest, stimulate its proliferation. COS cells are widely available, as are other suitable host cells. The clonally expanded autologous CD8⁺ T lymphocytes then are administered to the subject. The CD8⁺ T lymphocytes then stimulate the subject's immune response, thereby achieving the desired therapeutic goal.

Another method for selecting antigen-specific CTL clones has recently been described (Altman et al., Science 274:94-96, 1996; Dunbar et al., Curr. Biol. 8:413-416, 1998), in which fluorogenic tetramers of MHC class I molecule/peptide complexes are used to detect specific CTL clones. Briefly, soluble MHC class I molecules are folded in vitro in the presence of β₂-microglobulin and a peptide antigen which binds the class I molecule. After purification, the MHC/peptide complex is purified and labeled with biotin. Tetramers are formed by mixing the biotinylated peptide-MHC complex with labeled avidin (e.g. phycoerythrin) at a molar ratio or 4:1. Tetramers are then contacted with a source of CTLs such as peripheral blood or lymph node. The tetramers bind CTLs which recognize the peptide antigen/MHC class I complex. Cells bound by the tetramers can be sorted by fluorescence activated cell sorting to isolate the reactive CTLs. The isolated CTLs then can be expanded in vitro for use as described herein.

To detail a therapeutic methodology, referred to as adoptive transfer (Greenberg, J. Immunol. 136(5): 1917, 1986; Riddel et al., Science 257: 238, 1992; Lynch et al, Eur. J. Immunol. 21: 1403-1410, 1991; Kast et al., Cell 59: 603-614, 1989), cells presenting the desired complex are combined with CTLs leading to proliferation of the CTLs specific thereto. The proliferated CTLs are then administered to a subject with a cellular abnormality which is characterized by certain of the abnormal cells presenting the particular complex. The CTLs then lyse the abnormal cells, thereby achieving the desired therapeutic goal.

The foregoing therapy assumes that at least some of the subject's abnormal cells present the relevant HLA/peptide complex. This can be determined very easily, as the art is very familiar with methods for identifying cells which present a particular HLA molecule, as well as how to identify cells expressing DNA of the pertinent sequences, in this case a tumor associated gene sequence. Once cells presenting the relevant complex are identified by screening, they can be combined with a sample from a patient, where the sample contains CTLs. If the HLA/peptide complex presenting cells are lysed by the mixed CTL sample, then it can be assumed that a tumor associated gene derived peptide (e.g., TRA) is being presented, and the subject is an appropriate candidate for the therapeutic approaches set forth herein.

Adoptive transfer is not the only form of therapy that is available in accordance with the invention. CD8⁺ T lymphocytes can also be provoked in vivo, using a number of approaches. One approach is the use of non-proliferative cells expressing the complex. The cells used in this approach may be those that normally express the complex, which could be dendritic cells or cells transfected with one or both of the genes necessary for presentation of the complex. Chen et al., (Proc. Natl. Acad. Sci. USA 88: 110-114, 1991) exemplifies this approach, showing the use of transfected cells expressing HPV-E7 peptides in a therapeutic regime. Various cell types may be used. Similarly, vectors carrying one or both of the genes of interest may be used. Viral or bacterial vectors are especially preferred. For example, nucleic acids which encode a MAGE-A1 and/or a MAGE-A3 HLA binding peptide may be operably linked to promoter and enhancer sequences which direct expression of the MAGE-A1 and/or MAGE-A3 HLA binding peptide in certain tissues or cell types. The nucleic acid may be incorporated into an expression vector. Expression vectors may be unmodified extrachromosomal nucleic acids, plasmids or viral genomes constructed or modified to enable insertion of exogenous nucleic acids, such as those encoding MAGE-A1 and/or MAGE-A3 HLA binding peptides. Nucleic acids encoding a MAGE-A1 and/or a MAGE-A3 HLA binding peptide also may be inserted into a retroviral genome, thereby facilitating integration of the nucleic acid into the genome of the target tissue or cell type. In these systems, the gene of interest is carried by a microorganism, e.g., a Vaccinia virus, retrovirus or the bacteria BCG, and the materials de facto “infect” host cells. The cells which result present the complex of interest, and are recognized by autologous CD8⁺ T cells, which then proliferate.

A similar effect can be achieved by combining a MAGE-A1 and/or a MAGE-A3 HLA binding peptide with an adjuvant to facilitate incorporation into HLA class I presenting cells in vivo. If larger than the HLA class I binding portion, the MAGE-A1 and/or MAGE-A3 the HLA binding peptide can be processed if necessary to yield the peptide partner of the HLA molecule while the TRA is presented without the need for further processing. Generally, subjects can receive an intradermal injection of an effective amount of the MAGE-A1 and/or the MAGE-A3 immunogenic polypeptide. Initial doses can be followed by booster doses, following immunization protocols standard in the art.

As part of the immunization protocols, substances which potentiate the immune response may be administered with nucleic acid or peptide components of a cancer vaccine. Such immune response potentiating compound may be classified as either adjuvants or cytokines. Adjuvants may enhance the immunological response by providing a reservoir of antigen (extracellularly or within macrophages), activating macrophages and stimulating specific sets of lymphocytes. Adjuvants of many kinds are well known in the art; specific examples include MPL (SmithKline Beecham), a congener obtained after purification and acid hydrolysis of Salmonella minnesota Re 595 lipopolysaccharide, saponins such as QS21 (SmithKline Beecham), a pure QA-21 saponin purified from Quillja saponaria extract, DQS21, described in PCT application WO96/33739 (SmithKline Beecham), QS-7, QS-17, QS-18, and QS-L1 (So et al., Mol. Cells 7:178-186, 1997); incomplete Freund's adjuvant; complete Freund's adjuvant; montanide; immunostimulatory oligonucleotides (see e.g. CpG oligonucleotides described by Kreig et al., Nature 374:546-9, 1995); vitamin E and various water-in-oil emulsions prepared from biodegradable oils such as squalene and/or tocopherol. Preferably, the peptides are administered mixed with a combination of DQS21/MPL. The ratio of DQS21 to MPL typically will be about 1:10 to 10:1, preferably about 1:5 to 5:1 and more preferably about 1:1. Typically for human administration, DQS21 and MPL will be present in a vaccine formulation in the range of about 1 μg to about 100 μg. Other adjuvants are known in the art and can be used in the invention (see, e.g. Goding, Monoclonal Antibodies: Principles and Practice, 2nd Ed., 1986). Methods for the preparation of mixtures or emulsions of peptide and adjuvant are well known to those of skill in the art of vaccination.

Other agents which stimulate the immune response of the subject can also be administered to the subject. For example, other cytokines are also useful in vaccination protocols as a result of their lymphocyte regulatory properties. Many other cytokines useful for such purposes will be known to one of ordinary skill in the art, including interleukin-12 (IL-12) which has been shown to enhance the protective effects of vaccines (see, e.g., Science 268:1432-1434, 1995), GM-CSF and IL-18. Thus cytokines can be administered in conjunction with antigens and adjuvants to increase the immune response to the antigens.

There are a number of additional immune response potentiating compounds that can be used in vaccination protocols. These include costimulatory molecules provided in either protein or nucleic acid form. Such costimulatory molecules include the B7-1 and B7-2 (CD80 and CD86 respectively) molecules which are expressed on dendritic cells (DC) and interact with the CD28 molecule expressed on the T cell. This interaction provides costimulation (signal 2) to an antigen/MHC/TCR stimulated (signal 1) T cell, increasing T cell proliferation and effector function. B7 also interacts with CTLA4 (CD152) on T cells and studies involving CTLA4 and B7 ligands indicate that the B7-CTLA4 interaction can enhance antitumor immunity and CTL proliferation (Zheng et al., Proc. Nat'l Acad. Sci. USA 95:6284-6289, 1998).

B7 typically is not expressed on tumor cells so they are not efficient antigen presenting cells (APCs) for T cells. Induction of B7 expression would enable the tumor cells to stimulate more efficiently CTL proliferation and effector function. A combination of B7/IL-6/IL-12 costimulation has been shown to induce IFN-gamma and a Th1 cytokine profile in the T cell population leading to further enhanced T cell activity (Gajewski et al., J. Immunol. 154:5637-5648, 1995). Tumor cell transfection with B7 has been discussed in relation to in vitro CTL expansion for adoptive transfer immunotherapy by Wang et al. (J. Immunother. 19:1-8, 1996). Other delivery mechanisms for the B7 molecule would include nucleic acid (naked DNA) immunization (Kim et al., Nature Biotechnol. 15:7:641-646, 1997) and recombinant viruses such as adeno and pox (Wendtner et al., Gene Ther. 4:726-735, 1997). These systems are all amenable to the construction and use of expression cassettes for the coexpression of B7 with other molecules of choice such as the antigens or fragment(s) of antigens discussed herein (including polytopes) or cytokines. These delivery systems can be used for induction of the appropriate molecules in vitro and for in vivo vaccination situations. The use of anti-CD28 antibodies to directly stimulate T cells in vitro and in vivo could also be considered. Similarly, the inducible co-stimulatory molecule ICOS which induces T cell responses to foreign antigen could be modulated, for example, by use of anti-ICOS antibodies (Hutloff et al., Nature 397:263-266, 1999).

Lymphocyte function associated antigen-3 (LFA-3) is expressed on APCs and some tumor cells and interacts with CD2 expressed on T cells. This interaction induces T cell IL-2 and IFN-gamma production and can thus complement but not substitute, the B7/CD28 costimulatory interaction (Parra et al., J. Immunol., 158:637-642, 1997; Fenton et al., J. Immunother., 21:95-108, 1998).

Lymphocyte function associated antigen-1 (LFA-1) is expressed on leukocytes and interacts with ICAM-1 expressed on APCs and some tumor cells. This interaction induces T cell IL-2 and IFN-gamma production and can thus complement but not substitute, the B7/CD28 costimulatory interaction (Fenton et al., 1998). LFA-1 is thus a further example of a costimulatory molecule that could be provided in a vaccination protocol in the various ways discussed above for B7.

Complete CTL activation and effector function requires Th cell help through the interaction between the Th cell CD40L (CD40 ligand) molecule and the CD40 molecule expressed by DCs (Ridge et al., Nature 393:474, 1998; Bennett et al., Nature 393:478, 1998; Schoenberger et al., Nature 393:480, 1998). This mechanism of this costimulatory signal is likely to involve upregulation of B7 and associated IL-6/IL-12 production by the DC (APC). The CD40-CD40L interaction thus complements the signal 1 (antigen/MHC-TCR) and signal 2 (B7-CD28) interactions.

The use of anti-CD40 antibodies to stimulate DC cells directly, would be expected to enhance a response to tumor associated antigens which are normally encountered outside of an inflammatory context or are presented by non-professional APCs (tumor cells). In these situations Th help and B7 costimulation signals are not provided. This mechanism might be used in the context of antigen pulsed DC based therapies or in situations where Th epitopes have not been defined within known tumor associated antigen precursors.

When administered, the therapeutic compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents. The term “pharmaceutically acceptable” means a non-toxic material that does not interfere with the effectiveness of the biological activity of the active ingredients. The characteristics of the carrier will depend on the route of administration.

The therapeutics of the invention can be administered by any conventional route, including injection or by gradual infusion over time. The administration may, for example, be oral, intravenous, intraperitoneal, intramuscular, intranasal, intracavity, subcutaneous, intradermal or transdermal.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The invention also contemplates gene therapy. The procedure for performing ex vivo gene therapy is outlined in U.S. Pat. No. 5,399,346 and in exhibits submitted in the file history of that patent, all of which are publicly available documents. In general, it involves introduction in vitro of a functional copy of a gene into a cell(s) of a subject which contains a defective copy of the gene, and returning the genetically engineered cell(s) to the subject. The functional copy of the gene is under operable control of regulatory elements which permit expression of the gene in the genetically engineered cell(s). Numerous transfection and transduction techniques as well as appropriate expression vectors are well known to those of ordinary skill in the art, some of which are described in PCT application WO95/00654. In vivo gene therapy using vectors such as adenovirus also is contemplated according to the invention.

The preparations of the invention are administered in effective amounts. An effective amount is that amount of a pharmaceutical preparation that alone, or together with further doses, stimulates the desired response. In the case of treating cancer, the desired response is inhibiting the progression of the cancer. This may involve only slowing the progression of the disease temporarily, although more preferably, it involves halting the progression of the disease permanently. In the case of inducing an immune response, the desired response is an increase in antibodies or T lymphocytes which are specific for the MAGE-A1 and/or MAGE-A3 immunogen(s) employed. These desired responses can be monitored by routine methods or can be monitored according to diagnostic methods of the invention discussed herein.

Where it is desired to stimulate an immune response using a therapeutic composition of the invention, this may involve the stimulation of a humoral antibody response resulting in an increase in antibody titer in serum, a clonal expansion of cytotoxic lymphocytes, or some other desirable immunologic response. It is believed that doses of immunogens ranging from one nanogram/kilogram to 100 milligrams/kilogram, depending upon the mode of administration, would be effective. The preferred range is believed to be between 500 nanograms and 500 micrograms per kilogram. The absolute amount will depend upon a variety of factors, including the material selected for administration, whether the administration is in single or multiple doses, and individual patient parameters including age, physical condition, size, weight, and the stage of the disease. These factors are well known to those of ordinary skill in the art and can be addressed with no more than routine experimentation.

### EXAMPLES

### Example 1:

### Identification of a MAGE-A1 Peptide Presented by HLA-B35

A method which selects naturally-processed peptides was used to identify CTL epitopes. CD8⁺ T cells, obtained from an individual without cancer, were stimulated with autologous dendritic cells infected with a recombinant Canarypox virus, ALVAC, containing the coding sequence of MAGE-A1 (SEQ ID NO:1; the amino acid sequence of MAGE-A1 protein is provided as SEQ ID NO:2; these sequences correspond to GenBank accession number NM_{—}004988). Responder cell microcultures, which specifically lysed autologous EBV-transformed B cells infected with vaccinia-MAGE-A1, were cloned by limiting dilution using autologous stimulator cells infected with a recombinant Yersinia-MAGE-A1. An anti-MAGE-A1 CTL clone was obtained and the epitope was found to be nonapeptide EADPTGHSY (SEQ ID NO:8; MAGE-Ab 1₆₁₋₁₆₉) presented by HLA-B35 molecules. About 14% of Caucasians express HLA-B3501 or B3503. The CTL clone lysed HLA-B35 tumor cells expressing MAGE-A1. Interestingly, peptide EADPTGHSY (SEQ ID NO:8) was already identified as a MAGE-A1 epitope recognized on HLA-A1 molecules by MZ2-CTL 82/30 (see U.S. Pat. No. 5,925,729).

I. Processing of Human Blood

Blood samples were processed as described in Chaux et al., J. Immunol. 163:2928-2936, 1999, with minor modifications.

Blood cells were collected as buffy-coat preparations from hemochromatosis patients LB 1118, LB1801 and LB1137. Peripheral blood mononuclear cells (PBMC) were isolated by Lymphoprep (Nycomed, Oslo, Norway) density gradient centrifugation. To minimize contamination of PBMC with platelets, the preparation was first centrifuged at room temperature for 20 min at 160 g. After removal of the top 20-25 ml, which contained most of the platelets, the tubes were centrifuged at room temperature for 20 min at 350 g. The interphase containing the PBMC was harvested and then washed 3 times in cold phosphate buffer solution with 2 mM EDTA in order to eliminate the remaining platelets. To generate autologous dendritic cells, PBMC were depleted from T lymphocytes by resetting with sheep erythrocytes (BioMerieux, Marcy l'Etoile, France) previously treated with 2-aminoethylisothiouronium (Sigma, St. Louis, Mo.). The lymphocyte-depleted PBMC were left to adhere for 1.5-2 h at 37° C. in Falcon culture flasks (Becton-Dickinson Labware, Meylan, France) at a density of 2-3×10⁶ cells/ml in RPMI 1640 medium (Life Technologies, Gaithersburg, Md.) supplemented with 0.24 mM L-asparagine, 0.55 mM L-arginine and 1.5 mM L-glutamine (AAG), and 10% FCS (hereafter referred to as complete RPMI medium). Nonadherent cells were discarded, and adherent cells were cultured in the presence of IL-4 (10 ng/ml) and GM-CSF (100 ng/ml) in complete RPMI medium. Cultures were fed on days 2 and 4 by replacing one-half of the medium with fresh medium plus IL-4 (10 ng/ml) and GM-CSF (100 ng/ml). On day 5, the nonadherent cell population was used as a source of enriched dendritic cells. Monocyte-derived dendritic cells (DC) were frozen on day 6 or 7.

Rosetted T cells were treated with NH₄Cl (160 mM) to lyse the sheep erythrocytes and then washed. CD8⁺ T lymphocytes were isolated from rosetted T cells by positive selection using an anti-CD8 mAb coupled to magnetic microbeads (Miltenyi Biotech, Bergisch Gladbach, Germany) and be sorting through a magnetic cell separation system, as recommended by the manufacturer. The lymphocytes were frozen and the thawed the day before the coculture with dendritic cells.

II. Cell Lines and Cytokines

The EBV-transformed B(EBV-B) cell lines and the melanoma cell lines were cultured in IMDM (Life Technologies, Gaithersburg, Md.) supplemented with 10% FCS (Life Technologies, AAG, 100 U/ml Penicillin and 100 μg/ml streptomycin. HeLa and COS-7 cells were maintained in DMEM (Life Technologies) supplemented with 10% FCS.

Human recombinant IL-2 was purchased from Eurocetus (Amsterdam, Netherlands). The concentration needed to obtain half maximum proliferation of mouse CTLL-2 cells is one U/ml of IL-2. IL-7 was purchased from Genzyme (Cambridge, Mass.), and GM-CSF was purchased from Schering-Plough (Brinny, Ireland). Human recombinant IL-4, IL-6, and IL-12 were produced in our laboratory. The concentration needed to obtain half maximum proliferation of mouse 7TD1 cells is 1 U/ml of IL-6 (Van Snick et al. J. Exp. Med. 165:641, 1987).

III. Generation of the Recombinant Viruses

The recombinant canarypox ALVAC-MAGE-A1 (vCP 299), vaccinia WR-MAGE-A1 (vP 1188 and vP 1264), and the parental vaccinia (vP 1170) viruses were provided by Virogenetics (Troy, N.Y.). Retroviral vector M1-CSM encodes the full length MAGE-A1 protein in the truncated form of the human low affinity nerve growth factor receptor (ΔLNGFr). It was produced as previously reported (Mavilio, et al. Blood 83:1988, 1994). The parental WR strain of vaccinia (vP 1170) contained the parent vector pKILGPT of 2826 BP (Virogenetics, Troy, N.Y.). A sequence coding for MAGE-A1, placed after the vaccinia virus H6 promoter, was cloned into the pKILGPT vector, creating donor plasmid MAWO35.

The donor plasmid was transfected into CEF cells containing the genomic DNA of vaccinia strain WR, yielding the recombinant vaccinia virus WR-MAGE-A1, by way of in vivo recombination, and selected with BrdU and Xgal (Perkins et al., J. Virol. 63:3829-3836, 1989).

For preparation of recombinant canarypox, a MAGE-A1 coding sequence, placed after the vaccinia virus H6 promoter, was cloned into a pUC8-based vector to generate donor plasmid MAWO36. Recombinant ALVAC-MAGE-A1 virus was generated by using the donor plasmid MAWO36 and following well known procedures (Current Protocols and Molecular Cloning, Ausubel et al., John Wiley and Sons, New York, N.Y.; Ferrari et al., Blood 90:2406-2416, 1997). Briefly, a DNA fragment containing the complete MAGE-A1 ORF was generated and linked to the vaccinia H6 promoter by standard PCR procedures. The H6/MAGE-A1 DNA segment was subcloned into a plasmid such that the H6/MAGE-A1 DNA segment was flanked by ALVAC DNA from the C3 insertion site of the ALVAC genome. Thus the organization of the elements in the plasmid was as follows: ALVAC C3 left flanking arm, H6/MAGE-A1, ALVAC right flanking arm. The ALVAC-MAGE-A1 recombinant virus was generated by in vivo recombination between the plasmid described above and ALVAC genomic DNA following standard procedures. Recombinant virus was selected by hybridization with MAGE-A1 -specific DNA probes and plaque purified. The resulting ALVAC-MAGE-A3 recombinant was designated vCP 299. Expression analysis with MAGE-A1 -specific antisera confirmed the expression of MAGE-A1 polypeptides in cells infected with vCP 299.

EBV-B cells or PHA-activated T cells were transduced by coculture with irradiated packaging cell lines producing the M1 -CSM vector in the presence of polybrene (8 μg/ml). After 72 h, lymphocytes were harvested and seeded in fresh medium. The percentage of infected cells was evaluated 48 h later by flow cytometry for LNGFr expression with the mAb 20.4 (American Type Culture Collection, Manassas, Va.). The LNGFr-positive cells were purified by magnetic cell sorting using rat anti-mouse IgG1-coated beads (Dynabeads M-450, Dynal, Oslo, Norway).

IV. Generation of the Yersinia-MAGE-A1 Construct

The wild-type Yersinia enterocolitica is an extracellular bacteria causing gastrointestinal syndromes in humans. Y enterocolitica adhere to the surface of target cells and possess a virulence apparatus called the Yop virulon, which enables the translocation of toxic effector proteins, including YopE, into the cytosol of the host cell. A new strain, MRS40 (pABL 403), has recently been constructed, where the genes encoding toxic Yop proteins are mutated or truncated (Boland et al., Infect. Immun. 66:1878, 1998). Interestingly, this polymutant strain maintains its ability to translocate proteins in fusion with a truncated YopE into the cytosol of eukaryotic cells, but it does not elicit cytotoxicitity and can therefore be used as a vector to inject a protein into the cytosol of eukaryotic cells.

The sequence encoding protein MAGE-A1 was inserted in frame with a sequence encoding a truncated YopE, YopE,₁₋₁₃₀, containing the first 130 amino acids of YopE. The open reading frame of MAGE-A1 was amplified by PCR using a MAGE-A1 cDNA cloned in pcDNAI/Amp (Invitrogen, Groningen, The Netherlands) as the template. The upstream primer, AAACTGCAGATGTCTCTTGAGCAGAGGAGTC (SEQ ID NO:3), consisted of the first nucleotides of the open reading frame of MAGE-A1 preceded by a PstI site. The downstream primer, AAACTGCAGTCAGACTCCCTCTTCCTCCTC (SEQ ID NO:4), consisted of nucleotides complementary to the last nucleotides of the open reading frame of MAGE A1 followed by a PstI site. The PCR product was digested with PstI and inserted in frame with the truncated YopE at the PstI site of vector pMS 111 (Sory et al., Mol. Microbiol. 14:583, 1994). pMS111-MAGE A1 was electroporated in Escherichia coli strain DH5αF'IQ. The DNA of a recombinant clone was sequenced and then electroporated in E. coli strain SM10. Plasmid pMS111-MAGE A1 was mobilized by SM10into Y enterocolitica MRS40. Recombinant MRS40 were selected on agar-containing medium and supplemented with nalidixic acid (35 μg/ml), sodium m-arsenite (1 mM), and chloramphenicol (12 μg/ml) (Neyt et al., J. Bacteriol. 179:612, 1997).

One colony of Y. enterocolitica MRS40 containing pMS111-MAGE-A1 was then grown overnight at 28° C. in Luria-Bertani medium supplemented with nalidixic acid (35 μg/ml), sodium m-arsenite (1 mM) and chloramphenicol (12 μg/ml). To obtain an optical density of 0.2 at 600 nm, this culture is diluted and cultured at 28° C. for about 2 h. The bacteria were then washed in 0.9% NaCl and resuspended at 10⁸/ml in 0.9% NaCl, assuming that a culture giving an OD₆₀₀ equal to 1 contains 5×10⁸ bacteria/ml. Irradiated EBV-B cells were resuspended at 10⁶ in 3.8 ml of RPMI without antibiotics, and supplemented with 10% FCS and AAG. Then, 200 μl of the bacterial suspension were added. Two hours after infection, gentamicin (30 μg/ml) was added for the next 2 h. The cells were finally washed three times before use as stimulator cells.

V. Mixed CD8⁺ Lymphocytes and ALVAC-MAGE-A1 -dendritic Cells Culture

Dendritic cells (3×10⁶) were thawed and infected with ALVAC-MAGE-A1 at a multiplicity of infection (MOI) of 30 in 1 ml of complete RPMI medium at 37° C. under 5% CO₂. The infected dendritic cells were washed after 2 h. Autologous responder CD8⁺ T lymphocytes (1.5×10⁵) were mixed with infected dendritic cells (3×10⁴) in microwells in 200 μl of complete IMDM in the presence of IL-6 (1000 U/ml) and IL-12 (10 ng/ml) plus 15 μg/ml of gentamycin. On days 7 and 14, autologous dendritic cells that were either thawed or cultured for 5 days were infected with ALVAC-MAGE-A1 and used to restimulate the CD8⁺ lymphocytes in medium supplemented with IL-2 (10 U/ml) and IL-7 (5 ng/ml) plus 15 μg/ml of gentamycin. The responder CD8⁺ T cells were assessed on day 21 or day 28 for their capacity to lyse autologous EBV-B cells infected with vaccinia-MAGE-A1.

VI. Cytoxicity Assay

Before infection of EBV-B targets, the vaccinia samples were sonicated for 30 sec. The cytoxicity of an aliquot of each microculture was tested on autologous EBV-B cells infected with vaccinia-MAGE-A1 or control vaccinia. Infection was performed on 2×10⁶ target cells for 2 h at a MOI of 20 in 500 μl of complete RPMI medium. Infected cells were washed, labeled with 100 μCi of Na(⁵¹Cr)O₄, and added to the responder cells at an E:T ratio of about 40:1. Unlabeled K562 cells were also added (5×10⁴ per V-bottom microwell) to block NK activity. Individual microcultures were tested on each target in duplicate. Chromium release was measured after incubation at 37° C. for 4 h.

VII. Obtention of CTL Clones Specific for MAGE-A1

Microculture 533/A3 containing cells that specifically lysed autologous EBV-B cells infected with the vaccinia-MAGE-A1 construct was cloned by limiting dilution using for the first two weekly stimulations irradiated autologous EBV-B cells infected with Yersinia-MAGE-A1 as stimulating cells, irradiated LG2-EBV cells as feeder cells, IL-2 (50 U/ml) and 15 μg/ml of gentamycin. CTL clones were then maintained in culture by alternate weekly restimulations with PHA (0.5 μg/ml), feeder cells and IL-2, or with Yersinia-MAGE-A1 infected EBV-B cells as stimulating cells, feeder cells, IL-2 and IL-12 (5 ng/ml).

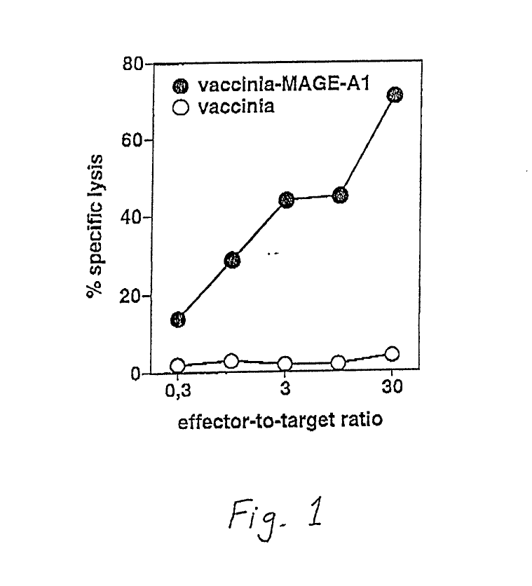

The clones were tested for specific lysis of autologous EBV-B cells infected with the vaccinia-MAGE-A1 construct, or with the parental vaccinia as a negative control (FIG. 1). EBV-B cells were infected for 2 h with the vaccinia vectors at a multiplicity of infection of 20, ⁵¹Cr-labeled for 1 h and incubated with the CTL at the indicated effector-to-target ratios. Chromium release was measured after 4 h. Experiments were performed in triplicate. Clone LB 1841 533/A7 (hereafter referred to as clone 7) was found positive and was used in the next experiments.

VIII. The MAGE-A1 Epitope is Presented to CTL by HLA-B35 Molecules

Donor LB 1841 was typed HLA-A3, -B35, -B60, -Cw3, and -Cw4. To identify the HLA molecule that presents the MAGE-A1 epitope recognized by CTL clone 7, COS-7 cells were transfected with the MAGE-A1 cDNA together with cDNAs coding for putative HLA presenting molecules. For HLA-B35, both HLA-B3501 and B3503 coding cDNAs were tested in parallel. HLA-B*4001 is one of the HLA-alleles comprised in the serological type HLA-B60.

The HLA coding sequences were isolated from various individuals; in particular, the HLA-B3501 cDNA was obtained by RT-PCR using RNA from patient LB 1074 as template and was inserted in pcDNA3. The HLA-B3503 cDNA was obtained by RT-PCR using RNA from patient BB49 as template. This PCR product cDNA was inserted in pcDNA3. The other cDNAs were inserted in expression vector pcDNA3 or pcD-SRα.

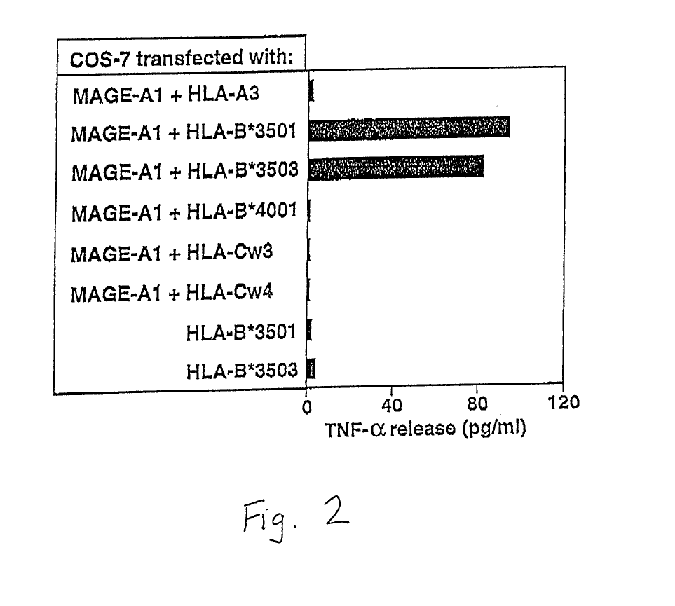

In brief, COS-7 cells were cotransfected by the DEAE-dextran procedure. Transfections were performed in microwells with 18,000 COS-7 cells, 25 ng HLA cDNA and 50 ng MAGE-A1 cDNA in the presence of 800 μg DEAE dextran and 200 μM chloroquine. The transfected COS-7 cells were incubated 24 hours at 37° C. and 8% CO₂. One day after transfection, the transfectants were tested for their ability to stimulate the production of TNF by clone 7. Briefly, 2,000 CTL clone 7 were added to the microwells containing the transfectants, in a total volume of 100 μl of Iscove's complete medium containing 25 U/ml of IL-2. After 24 hours, the supernatant was collected and its TNF content was determined by testing its cytotoxic effect on cells of WEHI-164 clone 13 in a MTT colorimetric assay as previously described (Hansen et al., J. Immunol. Methods 119:203-210, 1989; Traversari et al., Immunogenetics 35:145-152, 1992). Experiments were performed in triplicate. The cells transfected with HLA-B3501 or HLA-B3503, in combination with MAGE-A1, stimulated CTL clone 7 to produce TNF (FIG. 2).

In additional experiments, COS-7 cells were transfected with HLA-B3503 and either MAGE-A2, -A3, -A4, -A6, -A8, -A9, -A10, -A11, -A12, -B1, -B2, -C1 or -C2, and then were tested for CTL lysis as described above. None of the transfected cells were able to stimulate CTL clone 7 to produce TNF (data not shown).

IX. Determination of the Antigenic Peptide

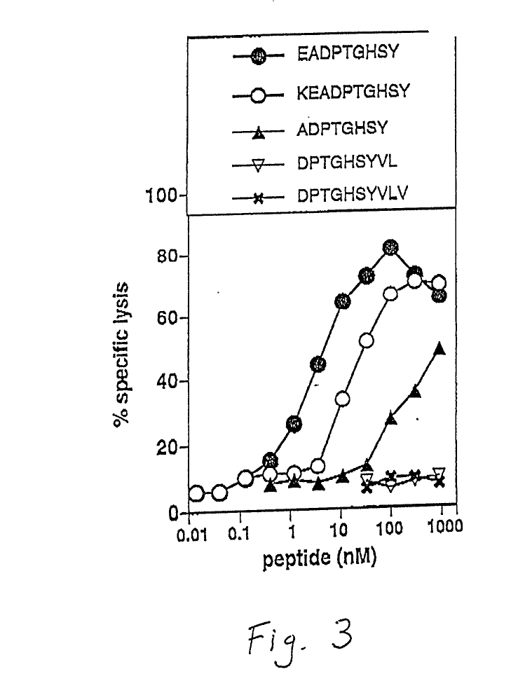

To identify the MAGE-A1 peptide recognized by CTL clone 7, a set of peptides of 12 amino acids, that overlapped by 8 amino acids and covered the entire MAGE-A1 protein sequence, was screened. Autologous EBV-B cells were distributed in microwells (10,000 cells per well), incubated with each of these peptides at a concentration of 2 μg/ml in 50 microliters, not washed and CTL clone 7 (2,000 CTLs per microwell) was added in 50 microliters. Recognition of the antigen by the CTLs was measured by the production of IFNγ in an ELISA assay using standard techniques. Peptide EADPTGHSYVLV (SEQ ID NO:5; MAGE-A1 amino acids 161-172) scored positive. The sequence of this peptide was screened for prediction of a HLA-B3501 binding peptide with the software available on the internet at http://bimas.dcrt.nih.gov/molbio/hla_bind/index.html. Peptides DPTGHSYVLV (SEQ ID NO:6) and DPTGHSYVL (SEQ ID NO:7) had the highest score. These peptides, as well as peptides EADPTGHSY (SEQ ID NO:8), KEADPTGHSY (SEQ ID NO:9) and ADPTGHSY (SEQ ID NO:10), were tested in a cytotoxicity assay with CTL clone 7 (FIG. 3).

EBV-B cells were ⁵¹Cr-labeled and incubated for 30 min with 3-fold dilutions of synthetic peptides EADPTGHSY (SEQ ID NO:8), KEADPTGHSY (SEQ ID NO:9) and ADPTGHSY (SEQ ID NO:10), DPTGHSYVL (SEQ ID NO:7) and DPTGHSYVLV (SEQ ID NO:6). Autologous CTL clone 7 was subsequently added at an effector-to-target ratio of 5:1. Chromium release was measured 4 h later. The concentrations indicated in the figure are the concentrations during the 4 h of incubation. Experiments were performed in triplicate.

Peptide EADPTGHSY (SEQ ID NO:8) was recognized and produced half-maximal lysis of autologous EBV-B target cells at ˜5-10 nM. Peptides KEADPTGHSY (SEQ ID NO:9) and ADPTGHSY (SEQ ID NO:10) were recognized less efficiently. Interestingly, peptide EADPTGHSY (SEQ ID NO:8) was already identified as a MAGE-A1 epitope recognized on HLA-A1 molecules by MZ2-CTL 82/30 (see U.S. Pat. No. 5,925,729).

X. Recognition by CTL Clone 7 of HLA-B35 Cells Expressing MAGE-A1

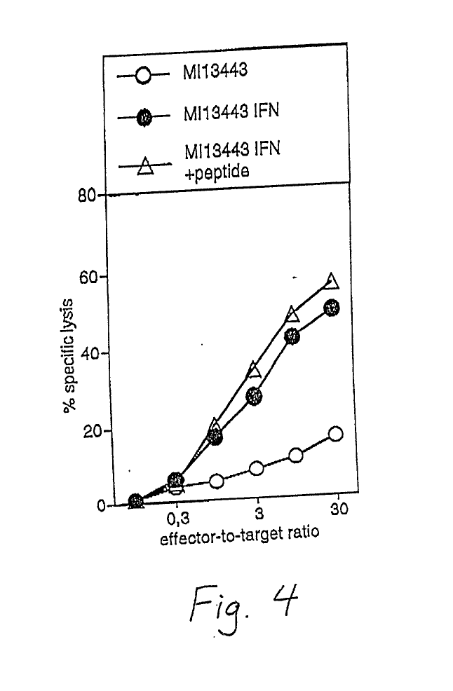

More importantly, CTL clone 7 was also able to lyse HLA-B35 melanoma cell line MI13443, which expressed MAGE-A1, but only when treated with 100 U/ml of IFNγ for 48 hours (FIG. 4).

MI13443 melanoma cells were ⁵¹Cr-labeled and incubated for 4 h with CTL clone 7 at the indicated effector-to-target ratios. Cell lines were either untreated (∘), pretreated with 100 U/ml IFNγ for 72h (●) or pulsed with 1 μg/ml of peptide EADPTGHSY (SEQ ID NO:8) (Δ). Experiments were performed in triplicate.

### Example 2:

### Identification of a MAGE-A1 Peptide Presented by HLA-B44

CD8⁺ T cells, obtained from an individual without cancer, were stimulated with autologous dendritic cells infected with a recombinant Canarypox virus, ALVAC, containing the coding sequence of MAGE-A1. Responder cell microcultures, which specifically lysed autologous EBV-transformed B cells infected with vaccinia-MAGE-A1, were cloned by limiting dilution using autologous stimulator cells infected with a recombinant Yersinia-MAGE-A1. An anti-MAGE-A1 CTL clone was obtained that recognized a MAGE-A1 epitope presented by HLA-B*4402 molecules. COS-7 cells, transfected with a HLA-B*4402 cDNA and a MAGE-A1 minigene that codes for amino acid sequence MEADPTGHSY (SEQ ID NO:14), are recognized by this new CTL clone, namely LB 1801 461/G4.2. Interestingly, peptide sequence MEADPTGHSY (SEQ ID NO:14) and its generic counterpart XEADPTGHSY (SEQ ID NO:53) contain EADPTGHSY (SEQ ID NO:8), which was identified as the MAGE-A1 peptide recognized on HLA-A1 molecules by MZ2-CTL 82/30 (see U.S. Pat. No. 5,925,729), and on HLA-B35 by LB1841 CTL533/A7 (see above). Importantly, the new CTL clone LB 1801 461/G4.2 lysed HLA-B*4402 tumor cells expressing MAGE-A1.

I. Processing of Human Blood

As described above, except that the interphase containing the PBMC was harvested and then washed three times in cold phosphate buffer solution without 2 mM EDTA.

II. Cytokines

As described above.

III. Generation of the Recombinant Viruses

As described above.

IV. Generation of the Yersinia-MAGE-A1 Construct

As described above.

V. Mixed CD8⁺ Lymphocytes and ALVAC-MAGE-A1 Dendritic Cells Culture

As described above, except that gentamycin was not added to the medium.

VI. Cytotoxicity Assay

As described above, except that the vaccinia samples were not sonicated.

VII. Obtention of CTL Clones Specific for MAGE-A1

Microculture 461/G4 containing cells that specifically lysed autologous EBV-B cells infected with the vaccinia-MAGE-A1 construct was stored frozen. The microculture was thawed, cultured overnight and restimulated with autologous monocyte derived dendritic cells infected with ALVAC-MAGE-A1 as described for the mixed CD8⁺ lymphocytes and ALVAC-MAGE-A1 -dendritic cells culture. The microculture was cloned one week after by limiting dilution using for the first two weekly stimulations irradiated autologous EBV-B cells infected with Yersinia-MAGE-A1 as stimulating cells, irradiated LG2-EBV as feeder cells, IL-2 (50 U/ml) and 15 μg/ml of gentamycin. CTL clones were then maintained in culture by weekly restimulations with PHA (0.5 μg/ml), feeder cells and IL-2. Once every two weeks 5 ng/ml IL-12 was added during restimulation.

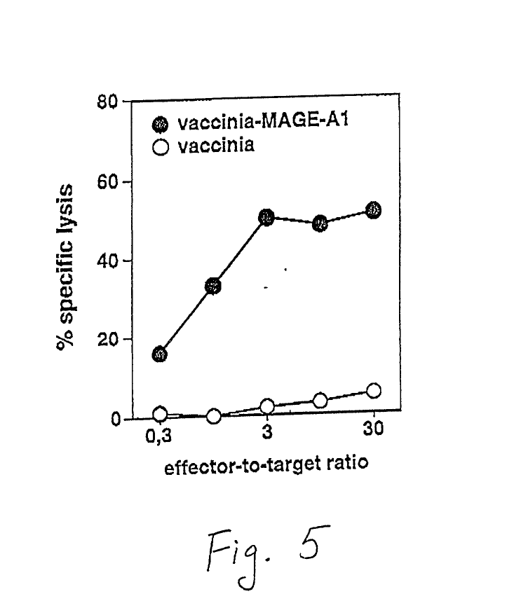

The clones were tested for specific lysis of autologous EBV-B cells infected with the vaccinia-MAGE-A1 construct, or with the parental vaccinia as a negative control. EBV-B cells were infected for 2 h with the vaccinia vectors at a multiplicity of infection of 20, ⁵¹Cr-labeled for 1 h and incubated with the CTL at the indicated effector-to-target ratios. Chromium release was measured after 4 h. Experiments were performed in triplicate. Clone LB 1801 461/G4.2 (hereafter referred to as clone 4.2) was found positive and was used in the next experiments (FIG. 5).

VIII. The MAGE-A1 Epitope is Presented to CTL by HLA-B*4402 Molecules

Donor LB 1801 was typed HLA-A2, -A28, -B*4402, -B53, -Cw4, and -Cw5. To identify the HLA molecule that presents the MAGE-A1 epitope to CTL clone 4.2, COS-7 cells were transfected with the MAGE-A1 cDNA together with cDNAs coding for putative HLA presenting molecules. In brief, 1.5×10⁴ COS-7 cells distributed in microwells were cotransfected with 50 ng of pcD-SRα-MAGE-A1 and 50 ng of plasmid containing coding sequences for HLA molecules in the presence of 1 μl per well of Lipofectamine. The HLA coding sequences were isolated from various individuals; in particular, the HLA-B*4402 cDNA was obtained by RT-PCR using RNA from patient LB33 as template. This PCR product was inserted in pcDNAI/amp.

The transfected COS-7 cells were incubated 24 hours at 37° C. and 8% CO₂. These transfectants were then tested for their ability to stimulate clone 4.2 to release TNF. Briefly, 1500 CTL were added to the microwells containing the transfectants, in a total volume of 100 μl of Iscove's complete medium containing 25 U/ml of IL-2. After 24 hours, the supernatant was collected and its TNF content was determined by testing its cytotoxic effect on cells of WEHI-164 clone 13 in a MTT colorimetric assay as previously described (Hansen et al., J. Immunol. Methods 119:203-210, 1989; Traversari et al., Immunogenetics 35:145-152, 1992). Experiments were performed in triplicate.

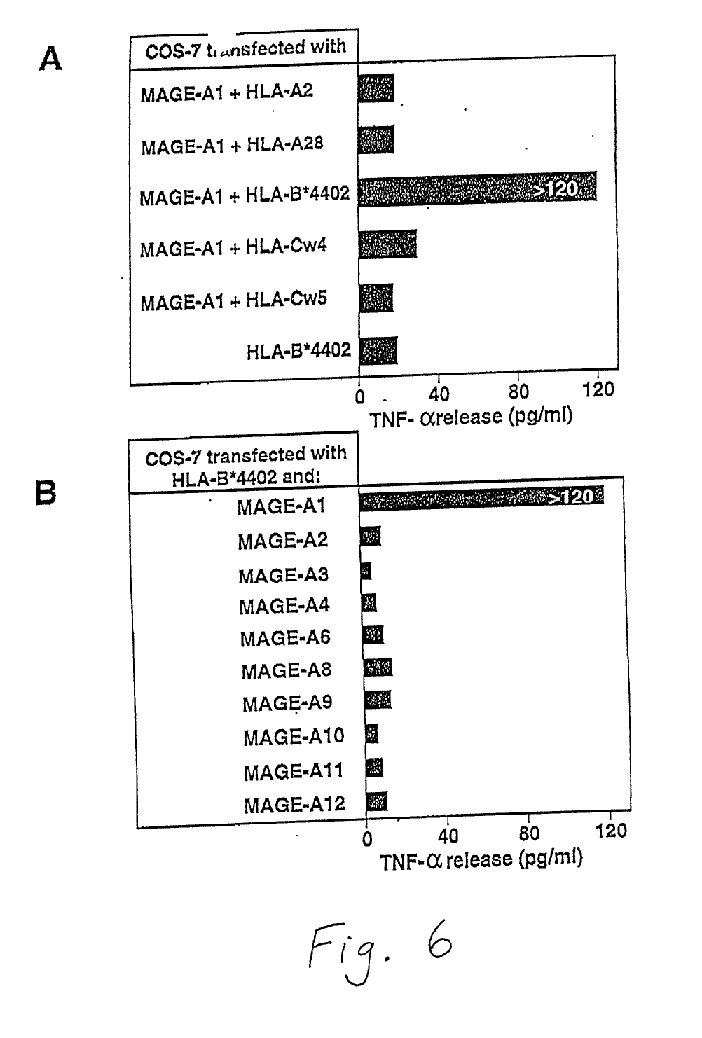

Monkey COS-7 cells were transiently transfected with a MAGE-A1 cDNA and each of the cDNAs coding for putative HLA-presenting molecules. The cDNAs were inserted in expression vector pcDNA3 or pcD-SRα. The cells transfected with HLA-B*4402, in combination with MAGE-A1, stimulated CTL clone 4.2 to produce TNF (FIG. 6A).

COS-7 cells were transiently transfected with the HLA-B*4402 cDNA and cDNA encoding different MAGE genes. One day after transfection, 1500 CTL 4.2 were added to the transfected cells. COS-7 cells transfected with HLA-B*4402 and either MAGE-A2, -A3, -A4, -A6, -A8, -A9, -A 10, -A11, -A12 were unable to stimulate CTL clone 4.2 to produce TNF (FIG. 6B).

IX. Determination of the MAGE-A1 cDNA Region Coding for the Antigenic Peptide

To identify the MAGE-A1 cDNA region that codes for the epitope that is presented by HLA-B*4402, truncated versions of the MAGE-A1 cDNA (minigenes) were generated by PCR, and the products were inserted in pcDNA3. COS-7 cells were transfected with each of the MAGE-A1 minigenes or the MAGE-A1 cDNA in combination with HLA-B*4402, and tested for recognition by CTL clone 4.2, as described under section VIII. In control experiments the COS-7 cells were transfected with a HLA-A1 cDNA together with the MAGE-A1 minigenes or the MAGE-A1 cDNA, and tested for recognition by the CTL82/30, which is reactive with MAGE-A1 peptide EADPTGHSY presented by HLA-A1.

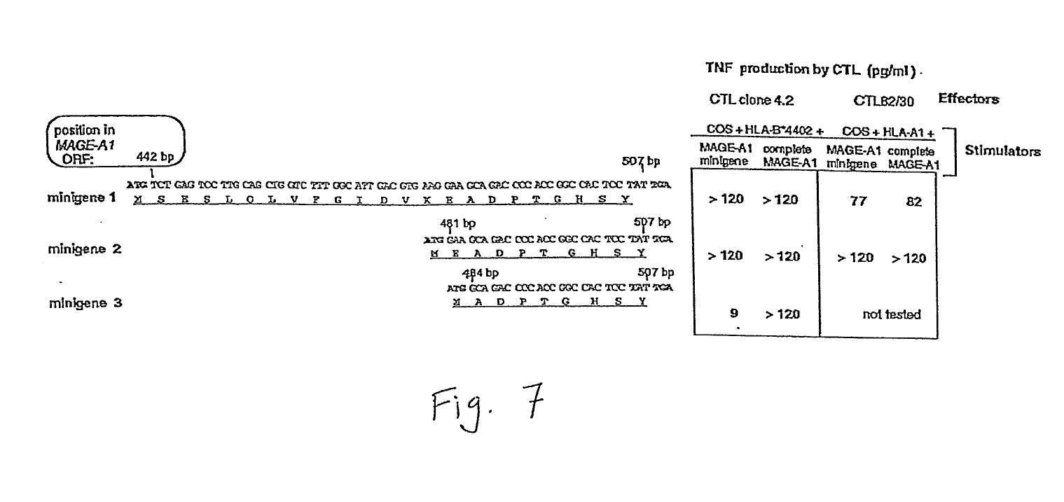

The coding region for the epitope was found to be located between nucleotides 442 and 507 of the MAGE-A1 cDNA (SEQ ID NO:1; nucleotide 1 is the A of the start codon), as determined by transfection of COS-7 with HLA-B*4402 and minigene 1(FIG. 7). Minigene 1 (SEQ ID NO:11) comprises nucleotides 442 to 507 of the MAGE-A1 cDNA joined to a 5′ ATG codon and a 3′ TGA codon. The peptide MSESLQLVFGIDVKEADPTGHSY (SEQ ID NO:12) is encoded by minigene 1. Two additional minigenes were also tested: minigene 2 (SEQ ID NO:13) which contains the sequence of nucleotides 481 to 507 of the MAGE-A1 cDNA joined to 5′ ATG and 3′ TGA codons and encodes the peptide MEADPTGHSY (SEQ ID NO:14); and minigene 3 (SEQ ID NO:15) which contains the sequence of nucleotides 484 to 507 of the MAGE-A1 cDNA joined to 5′ ATG and 3′ TGA codons and encodes the peptide MADPTGHSY (SEQ ID NO:16) (FIG. 7). COS-7 transfected with HLA-B*4402 and minigene 2, which codes for amino acid sequence MEADPTGHSY (SEQ ID NO:14), were recognized by CTL clone 4.2, whereas COS-7 transfected with HLA-B*4402 and minigene 3 were not recognized. Additional minigenes which encode peptides generically related to SEQ ID NO:14, having the sequence XEADPTGHSY (SEQ ID NO:53) where X is any amino acid, also are prepared and tested as described above. These additional minigenes also encode the MAGE-A1 epitope that is presented by HLA-B*4402.

Interestingly, minigene 2 was already identified to encode MAGE-A1 peptide EADPTGHSY (SEQ ID NO:8) recognized on HLA-A1 molecules by MZ2-CTL 82/30 (see U.S. Pat. No. 5,925,729), and on HLA-B35 by LB1841 CTL533/A7 as shown above in

### Example 1.

X. Recognition by CTL Clone 4.2 of HLA-B*4402 Cells Expressing MAGE-A1

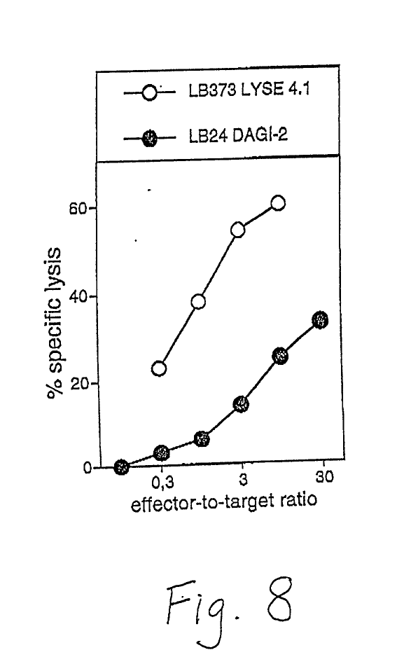

More importantly, CTL clone 4.2 was also able to lyse HLA-B*4402 melanoma cell lines that expressed MAGE-A1 : LB 373 LYSE 4.1 and LB 24 DAGI (FIG. 8). LB 373 LYSE and LB24 DAGI melanoma cells were ⁵¹Cr-labeled and incubated for 4 h with CTL clone 4.2 at the indicated effector-to-target ratios. Experiments were performed in triplicate.

### Example 3:

### Identification of MAGE-A3 Peptides Presented by HLA-B35

Gene MAGE-A3 is expressed in many different tumors, e.g., metastatic melanomas (76%), esophageal carcinomas (47%), head and neck tumors (49%), and bladder tumors (36%) (Van den Eynde and van der Bruggen, Curr. Opin. Immunology 9:684, 1997; and Francis Brasseur, personal communication). Recently, MAGE-A3 expression was also detected in 8 out of 27 patients with advanced (stage III) multiple myeloma (van Baren et al., Blood 94:1156, 1999). CTLs isolated from two melanoma patients, in mixed lymphocyte-tumor cell cultures, have been found to recognize MAGE-A3 peptides presented by HLA-A1, B37 (Gaugler et al., J. Exp. Med. 179:921, 1994; Tanzarella et al., Cancer Res. 59:2668, 1999). Synthetic peptides have been used to stimulate T cells from blood donors without cancer and this has led to the identification of MAGE-A3 epitopes presented by HLA-A1, A2, A24, and B44 (Celis et al., Proc. Natl. Acad. Sci. USA 91:2105, 1994; van der Bruggen et al., Eur. J. Immunol. 24:3038, 1994; Kawashima et al., Hum. Immunol. 59:1, 1998; Tanaka et al., Cancer Res. 57:4465,1997; Oiso et al., Int. J. Cancer 81:387, 1999; Herman et al., Immunogenetics 43:377, 1996). HLA-class II restricted peptides recognized by CD4+T cells have also been identified (Chaux et al., J. Exp. Med. 189:767, 1999; Manici et al., J. Exp. Med. 189:871, 1999). Presented herein is a new strategy for the identification of well processed antigenic peptides: dendritic cells transduced with gene MAGE-A3 were used as well stimulator cells for autologous T lymphocytes obtained from blood donors (Chaux et al., J. Immunol. 163:2928, 1999; Duffour et al., Eur. J. Immunol. 29:3329, 1999). A new MAGE-A3 epitope has been identified using this method and is presented herein.

I. Cell Lines, Media, Reagents.

The EBV-transformed B (EBV-B) cell lines and the melanoma cell lines were cultured in Iscove's modified Dulbecco medium (IMDM) supplemented with 10% fetal calf serum. 293-EBNA cells were maintained in DMEM medium supplemented with 10% fetal calf serum. All culture media were purchased from Life Technologies (Rockville, Md.) and supplemented with 0.24 mM L-asparagine, 0.55 mM L-arginine, 1.5 mM L-glutamine (AAG), 100 U/ml penicillin and 100 μg/ml streptomycin. Human recombinant IL-2 was purchased from Eurocetus (Amsterdam, The Netherlands). One unit/ml of IL-2 is the concentration needed to obtain half-maximal proliferation of mouse CTLL-2 cells. IL-7 was purchased from Genzyme (Cambridge, Ma.), GM-CSF (LEUCOMAX) was purchased from Novartis Pharma (Brussels, Belgium). IFN-γ was purchased from R&D Systems (Minneapolis, Minn.). Human recombinant IL-4, IL-6 and IL-12 were produced in our laboratory. One unit/ml of IL-6 is the concentration needed to obtain half-maximal proliferation of mouse 7TD1 cells (Van Snick et al., J. Exp. Med 165:641, 1987).

II. MAGE-A3 Sequences

The nucleotide sequence (SEQ ID NO:54) and amino acid sequence (SEQ ID NO:55) of MAGE-A3 were previously deposited as GenBank accession number NM_{—}005362.

III. Recombinant Viruses.

The recombinant canarypox viruses ALVAC-MAGE-A3 (vCP 1563) and ALVAC-βgal, and the parental vaccinia virus (vaccinia) (LVAR) were provided by Virogenetics (Troy, N.Y.). The vaccinia virus encoding MAGE-A3 (vaccinia MAGE-A3) was provided by Dr. Enzo Cerundolo (Oxford, UK). Before infection, the vaccinia virus and ALVAC samples were sonicated for 30 sec.

IV. Dendritic Cells and CD8⁺ Responder T Cells.

Blood samples were processed as described above, with minor modifications. Peripheral blood was obtained from hemochromatosis patient LB 1841 as standard buffy coat preparations. Peripheral blood mononuclear cells (PBMC) were isolated by centrifugation on Lymphoprep (Nycomed Pharma, Oslo, Norway). In order to minimize contamination of the PBMC by platelets, the preparation was first centrifuged at 1,000 rpm for 20 min at room temperature. After removal of the top 20-25 ml, containing most of the platelets, the tubes were centrifuged at 1,500 rpm for 20 min at room temperature. The interphase containing the PBMC was harvested and then washed 3 times (or more) in cold phosphate buffer solution with 2 mM EDTA in order to eliminate the remaining platelets. To generate autologous dendritic cells (DC), PBMC were depleted from T lymphocytes by rosetting with sheep erythrocytes (Bio Merieux, Marcy l'Etoile, France) treated with 2-aminoethylisothiouronium (Sigma-A1 drich, Bornem, Belgium). Rosetted T cells were treated with NH₄Cl (160 mM) to lyse the sheep erythrocytes and washed. CD8⁺ T lymphocytes were isolated from rosetted T cells by positive selection using an anti-CD8 monoclonal antibody coupled to magnetic microbeads (Miltenyi Biotech, Bergisch Gladbach, Germany). They were then sorted through a magnet and subsequently frozen. The day before the first stimulation, CD8⁺ T cells were thawed and grown overnight in IMDM supplemented with 10% human serum, AAG, and antibiotics (hereafter referred to as complete IMDM) in the presence of 10 U/ml of IL-2. The lymphocyte-depleted PBMC were left to adhere for 2 h at 37° C. in culture flasks (FALCON, Becton-Dickinson, Franklin Lakes, N.J.) at a density of 1×10⁶ cells/cm² in RPMI 1640 supplemented with HEPES (2.38 g/l), AAG, antibiotics, and 10% fetal calf serum (hereafter referred to as complete RPMI medium). Non-adherent cells were discarded and adherent cells were cultured in the presence of IL-4 (100 U/ml) and GM-CSF (100 ng/ml) in complete RPMI medium. Cultures were fed on day 2 and 4 by removing 1/3 of the volume and adding fresh medium with IL-4 (100 U/ml) and GM-CSF (100 ng/ml).

V. Mixed Lymphocyte-dendritic Cell Culture

Lymphocyte-dendritic cell cultures were performed as described above, with minor modifications. On day 0, autologous DC from donor LB 1841 (2×10⁶) were thawed and infected with the canarypox virus ALVAC-MAGE-A3, at a multiplicity of infection (MOI) of 10, in 200 μl of complete RPMI medium at 37° C. under 5% CO₂. The infected DC were washed after 2 h. Autologous responder CD8⁺ T lymphocytes (150,000) and infected DC (30,000) were co-cultured in U-bottomed microwells in 200 μl of complete IMDM in the presence of IL-6 (1,000 U/ml) and IL-12 (10 ng/ml). The CD8⁺ lymphocytes were restimulated once per week with autologous DC freshly infected with the canarypox ALVAC-MAGE-A3 construct, and grown in complete IMDM supplemented with IL-2 (10 U/ml) and IL-7 (5 ng/ml).

VI. Cytotoxicity Assay.

Cytotoxicity assays were performed as described above, with minor modifications. Autologous EBV-B cells were infected for 2 h with either the parental vaccinia virus (LVAR) or the vaccinia MAGE-A3 construct using an MOI of 20. The infected target cells were then labeled with Na(⁵¹Cr) O₄ for 1 h, washed, and added to the responder cells. Unlabeled K562 cells were also added (5×10⁴ per V-bottomed microwell) to block natural killer activity. Chromium release was measured after incubation at 37° C. for 4 h as described by Herin et al., Int. J. Cancer 39:390-396, 1987. The individual microcultures were tested in duplicate on each target at an effector-to-target ratio of approximately 1:1 -40:1.

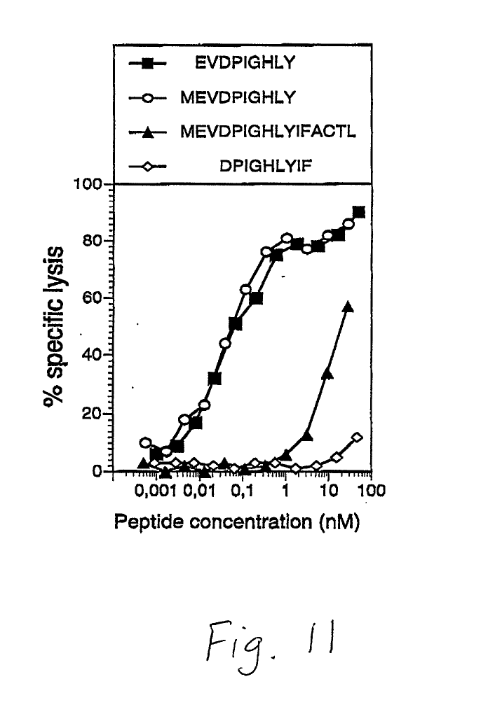

For cytotoxicity assays using peptide stimulation, EVB-B cells from patient LB 1841 were labeled with ⁵¹Cr and incubated for 10 min with 3-fold dilutions of synthetic peptides. Autologous CTL clone 41 was subsequently added at an effector-to-target ration of 5:1. Chromium release was measured after 4 h. The peptide concentrations indicated in FIG. 11 were the concentrations during the 4 h incubation.

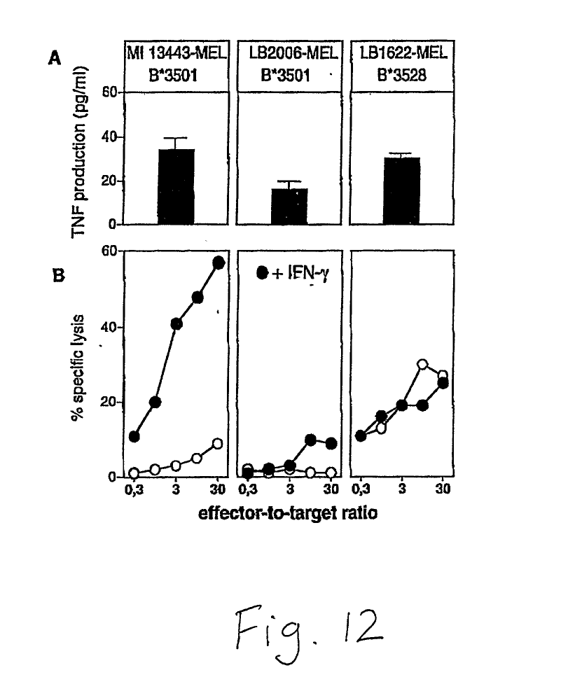

To determine recognition of HLA-B35 positive melanoma cell lines expressing MAGE-A3, TNF production and specific lysis assays were performed. The results are shown in FIG. 12A and FIG. 12B, respectively. Tumor cell lines were obtained from HLA-B35 patients; the B35 allele is indicated in FIG. 12. For TNF production, stimulator cells (20,000) were co-cultured in round microwells with 3,000 cells of CTL clone 41 and IL-2 (25U/ml). TNF production (FIG. 12A) was estimated after overnight culture, by the toxicity of the supernatants for TNF-sensitive WEHI 164-13 cells (Hansen et al., J. Immunol. Methods 119:203-210, 1989; Traversari et al., Immunogenetics 35:145-152, 1992). Error bars represent standard deviations of triplicate values. No TNF was measured in microwells containing either the tumor cells alone or the CTL alone. For cytotoxicity assays with HLA-B35 positive melanoma cell lines that expressed MAGE-A3, target cells were either untreated or pretreated with 100 U/ml IFN-γ for 48 h. Target cells were then ⁵¹Cr chromium labeled and incubated for 4 h with CTL clone 41 at various effector-to-target ratios as shown in FIG. 12B.

VII. Transfection of 293-EBNA Cells and IFN-γ-assay

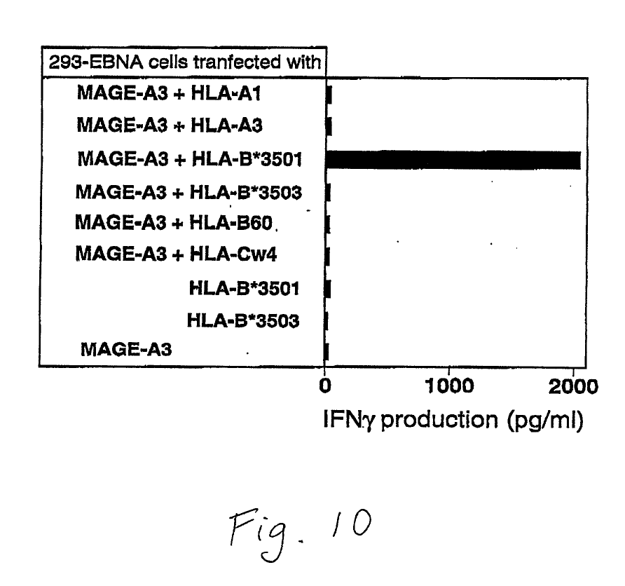

Transient transfection of 293-EBNA cells was performed with the LipofectAMINE™ reagent (GIBCO BRL, Life Technologies, Rockville, Md.). Briefly, 1.8×10⁴ 293-EBNA cells in a flat-bottomed 96-well dish were transfected with 50 ng of an expression plasmid containing the MAGE-A3 cDNA, 50 ng of an expression plasmid containing various HLA cDNAs (as shown in FIG. 10), and 1 μl of LipofectAMINE. The expression plasmids were either pcDNAI/Amp or pcDNA3. Transfected cells were tested in a CTL stimulation assay after 24 h. Briefly, 3000 LB1841-CTL 7 G6/41 cells were added to the transfected cells. IFN-γ production was determined by ELISA after overnight co-culture.

VIII. Identification of MAGE-A3 Peptides Presented by HLA-B35.

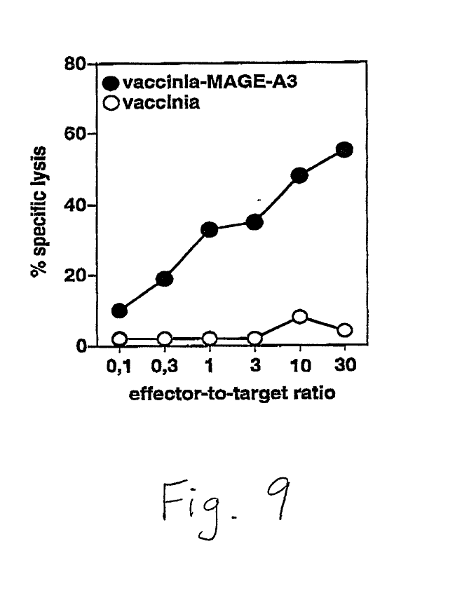

Briefly, monocyte-derived dendritic cells of blood donor LB 1841 were infected with an ALVAC engineered to contain the MAGE-A3 coding sequence (ALVAC-MAGE-A3). Infected dendritic cells were distributed in 96 microwells (3×10⁴ cells) and used to stimulate autologous CD8⁺ T lymphocytes (1.5×10⁵ cells), in the presence of IL-6 and IL-12. After two weeks restimulations with dendritic cells infected with ALVAC-MAGE-A3 in the presence of IL-2 and IL-7, responder cells were tested on day 21 for their specific lytic activity. The targets were autologous EBV-transformed B (EBV-B) cells infected with vaccinia-MAGE-A3, a vaccinia virus containing the MAGE-A3 coding sequence, or vaccinia virus (as a control) for 2 hours at a multiplicity of infection of 20. Infected cells were ⁵¹Cr labeled for 1 hour and incubated with the autologous CTL at effector-to-target ratios of 1, 3, 10 or 30. Chromium release was measured after 4 h. CTL clone LB 1841 526/G6.41 (CTL 41) was obtained by limiting dilution of microcultures with anti-MAGE-A3 reactivity. Stimulator cells were autologous EBV-B cells transduced with a retrovirus containing the MAGE-A3 coding sequence. CTL 41 specifically lysed EBV-B cells infected with vaccinia-MAGE-A3 (FIG. 9).

Blood donor LB 1841 was serologically typed HLA-A3, B*3501, B60, Cw3 and Cw4. To identify the HLA molecule that presents the MAGE-A3 epitope to CTL 41, 293-EBNA cells were transiently transfected with the MAGE-A3 cDNA together with each of the cDNAs encoding the putative HLA presenting molecules and then tested for recognition by CTL 41. The cDNAs were inserted in expression vector pcDNAI/Amp or pcDNA3. Transfections were performed in microwells with 18,000 293-EBNA cells, 50 ng of the HLA cDNA and 50 ng of the MAGE-A3 cDNA in the presence of 1 μl of LipofectAMINE. One day after transfection, 3,000 LB 1 841-CTL 7 G6/41 were added to the transfected cells. IFN-γ production was estimated after overnight co-culture by ELISA. Only the cells transfected with MAGE-A3 and HLA-B*3501 stimulated CTL 41 to produce IFN-γ (FIG. 10).

To identify the MAGE-A3 peptide recognized by CTL 41, a set of overlapping peptides of 16 amino acids, each of which overlapped by 12 amino acids, covering the entire MAGE-A3 protein sequence was screened. Briefly, EBV-B cells from patient LB1841 were ⁵¹Cr labeled and incubated for 10 min with 3-fold dilutions of synthetic peptides. Autologous CTL clone 41 was subsequently added at an effector-to-target ratio of 5:1. Chromium release was measured after 4 h. The concentration indicated in FIG. 11 were the peptide concentrations during the 4 h of incubation.

Peptide MEVDPIGHLYIFACTL (MAGE-A3₁₆₇₋₁₈₂; SEQ ID NO:57) scored positive. The consensus anchor residues for HLA-B35 are P in position 2 and Y, F, M, L or I in position 9 (Rammensee, H. G., J. Bachmann, and S. Stevanovic. 1997. MHC Ligands and Peptide Motifs. Springer, N.Y.). Peptide DPIGHLYIF (SEQ ID NO:58) contained the consensus anchor residues, but this peptide proved incapable of sensitizing EBV-B cells to lysis by CTL 41. In contrast, peptides MEVDPIGHLY (SEQ ID NO:59) and EVDPIGHLY (SEQ ID NO:56) scored positive in this cytotoxicity assay and produced half-maximal lysis of autologous EBV-B target cells at ˜0.05 nM (FIG. 11). This half-maximal lysis peptide concentration is lower than with the other MAGE antigenic peptides which produce half-maximal lysis at peptide concentrations from ˜0.1 to ˜25 nM (Chaux et al., J. Immunol. 163:2928, 1999; Traversari et al., J. Exp. Med. 176:1453, 1992; van der Bruggen et al., Eur. J. Immunol. 24:2134, 1994; Luiten and van der Bruggen, Tissue Antigens 55:149, 2000). Thus, the epitope recognized by CTL 41 does not contain consensus anchor residues for HLA-B35. It would therefore not have been discovered by an approach based on candidate peptides chosen on the basis of their sequence and used for in vitro stimulation of T lymphocytes. Peptides MEVDPIGHLY (SEQ ID NO:59) and EVDPIGHLY (SEQ ID NO:56) are encoded by the MAGE-A3 gene but not by another MAGE gene.

Six HLA-B35 melanoma cell lines that expressed MAGE-A3 were tested for their ability to stimulate CTL 41 to produce TNF. Three were not recognized, even in the presence of the peptide EVDPIGHLY (SEQ ID NO:56) and after an IFN-γ treatment, known to upregulate the expression of HLA molecules. This suggested the down-regulation of HLA-B35 molecules or the expression of another HLA-B35 subtype unable to present the peptide to CTL 41. The three other cell lines were recognized by CTL 41 (FIG. 12A) Genetic typing of these positive cells was performed. LB1622-MEL cells expressed B*3528 and, whether treated with IFN-γ or not, were lysed only slightly by CTL 41 (FIG. 12B). MI 13443-MEL cells expressed B*3501 and were perfectly lysed, but only after 48 h of incubation with IFN-γ. LB2006-MEL cells also expressed B*3501 but were not lysed at all. This correlated with their poor stimulation of CTL 41 to produce TNF (FIG. 12A).

Interestingly, peptide EVDPIGHLY (SEQ ID NO:56) presented to CTL 41 by HLA-B35 molecules was previously identified as the MAGE-A3 epitope recognized on HLA-A1 molecules by MZ2-CTL 82/30 (Gaugler et al., J. Exp. Med. 179:921, 1994, see also U.S. Pat. No. 6,034,214).

The results infer also that the clinical use of peptides including EVDPIGHLY (SEQ ID NO:56) and recombinant viruses or recombinant DNA encoding these peptides can now be extended to HLA-B35 patients; such use is entirely unexpected from the known binding specificity of HLA-B35 molecules. The frequency of the HLA-B35 phenotype in the Caucasian population is 20% (Marsh, S. G. E., P. Parham, and L. D. Barber. 2000. The HLA FactsBook. Academic Press, London). At least 27 different HLA-B35 alleles have been identified and the most frequent are HLA-B*3501 (9%), B*3502 (5%) and B*3503 (3%) (Ragupathi et al., Tissue Antigens 46:24, 1995). HLA-B*3501 and B*3503 have an equivalent peptide anchor motif but no data on HLA-B*3502 is available yet (Marsh, S. G. E., P. Parham, and L. D. Barber. 2000. The HLA FactsBook. Academic Press, London).

The results indicate that CTL 41, isolated from a B*3501 individual, recognized the MAGE-A3 peptide on B*3501 and B*3528 HLA molecules, but not on B*3503 molecules. There is a high probability that CTLs obtained from a B*3503 individual recognize the same peptide on B*3503 molecules. Firstly, HLA-B*3501 and B*3503 have an equivalent peptide anchor motif (Rammensee, H. G., J. Bachmann, and S. Stevanovic. 1997. MHC Ligands and Peptide Motifs. Springer, N.Y.). Secondly, Herman et al. previously isolated from a B*4402 individual an anti-MAGE-A3 CTL strictly restricted by B*4402 and, from a B*4403 individual, CTL strictly restricted by B*4403 that recognized the same peptide (Herman et al., Immunogenetics 43:377-383, 1996; Herman et al., Tissue Antigens 53:111-121, 1999). The possibility that the B35 subtype specificity results from the selective binding of the peptide to one subtype cannot be excluded.

Other aspects of the invention will be clear to the skilled artisan and need not be repeated here. Each reference cited herein is incorporated by reference in its entirety.

The terms and expressions which have been employed are used as terms of description and not of limitation, and there is no intention in the use of such terms and expressions of excluding any equivalents of the features shown and described or portions thereof, it being recognized that various modifications are possible within the scope of the invention.

Except where explicitly described otherwise, terms used in the singular also are meant to embrace the plural, and vice versa.

We claim:

1. A method for diagnosing a disorder characterized by expression of MAGE-A 1 in a subject typed as HLA-B35 positive, comprising:
contacting a biological sample isolated from the subject with an agent that is specific for a MAGE-A1 HLA binding peptide which comprises SEQ ID NO:10, and
determining the interaction between the agent and the MAGE-A1 HLA binding peptide as a determination of the disorder.

2. The method of claim 1 wherein the MAGE-A1 HLA binding peptide is selected from the group consisting of (i) peptides which consist of a fragment of the amino acid sequence of SEQ ID NO:2, (ii) peptides which comprise the amino acid sequence of SEQ ID NO:8, and (iii) functional variants of the peptides of (i) and (ii).

3. A method for diagnosing a disorder characterized by expression of MAGE-A1 in a subject typed as HLA-B44 positive, comprising:
contacting a biological sample isolated from the subject with an agent that is specific for a MAGE-A1 HLA binding peptide which comprises SEQ ID NO:53, and
determining the interaction between the agent and the MAGE-A1 HLA binding peptide as a determination of the disorder.

4. The method of claim 3 wherein the MAGE-A1 HLA binding peptide is selected from the group consisting of (i) peptides which consist of a fragment of the amino acid sequence of SEQ ID NO:2, (ii) peptides which comprise the amino acid sequence of SEQ ID NO:14, and (iii) functional variants of the peptides of (i) and (ii).

5. A method for diagnosing a disorder characterized by expression of a complex of a MAGE-A1 HLA-B35 or HLA-B44 binding peptide and a HLA-B35 or HLA-B44 molecule, comprising:
contacting a biological sample isolated from a subject suspected of having the disorder with an agent that binds the complex; and
determining binding between the complex and the agent as a determination of the disorder.

6. The method of claim 5 wherein the MAGE-A1 HLA-B35 binding peptide is selected from the group consisting (i) peptides which consist of a fragment of the amino acid sequence of SEQ ID NO:2, (ii) peptides which comprise the amino acid sequence of SEQ ID NO:10, and (iii) functional variants of the peptides of (i) and (ii).

7. The method of claim 5 wherein the MAGE-A1 HLA-B44 binding peptide is selected from the group consisting (i) peptides which consist of a fragment of the amino acid sequence of SEQ ID NO:2, (ii) peptides which comprise the amino acid sequence of SEQ ID NO:53, and (iii) functional variants of the peptides of (i) and (ii).

8. A method for enriching selectively a population of T lymphocytes with T lymphocytes specific for a MAGE-A1 HLA binding peptide, comprising:
contacting a source of T lymphocytes which contains a population of T lymphocytes with an agent presenting a complex of a MAGE-A1 HLA-B35 binding peptide comprising SEQ ID NO:10 and a HLA-B35 molecule or a complex of a MAGE-A1 HLA-B44 binding peptide comprising SEQ ID NO:53 and a HLA-B44 molecule, in an amount sufficient to selectively enrich the population of T lymphocytes with the T lymphocytes specific for one of the complexes.

9. The method of claim 8, wherein the agent is selected from the group consisting of an antigen presenting cell which expresses a HLA-B35 molecule contacted with a MAGE-A1 protein or a HLA binding fragment thereof which comprises SEQ ID NO:10, and an antigen presenting cell which expresses a HLA-B44 molecule contacted with a MAGE-A1 protein or a HLA binding fragment thereof which comprises SEQ ID NO:53.

10. The method of claim 8 wherein the MAGE-A1 HLA-B35 binding peptide is selected from the group consisting of (i) SEQ ID NO:8, SEQ ID NO:9 and SEQ ID NO:10, and (ii) functional variants of the peptides of (i).

11. The method of claim 8 wherein the MAGE-A1 HLA-B44 binding peptide is selected from the group consisting of (i) SEQ ID NO:12 and SEQ ID NO:14, and (ii) functional variants of the peptides of (i).

12. A method for treating a subject, typed as HLA-B35 or HLA-B44 positive, having a disorder characterized by expression of MAGE-A1, comprising:
administering to the subject an amount of a MAGE-A1 HLA-B35 binding peptide comprising SEQ ID NO:10or a MAGE-A1 HLA-B44 binding peptide comprising SEQ ID NO:53 sufficient to ameliorate the disorder.

13. The method of claim 12, wherein the MAGE-A1 HLA-B35 binding peptide is selected from the group consisting of (i) peptides which consist of a fragment of the amino acid sequence of SEQ ID NO:2, (ii) peptides which comprise the amino acid sequence of SEQ ID NO:8, and (iii) functional variants of the peptides of (i) and (ii).

14. The method of claim 13, further comprising administering to the subject at least one isolated HLA binding peptide selected from the group consisting of (1) MAGE-A1 HLA class I binding peptides other than peptides comprising SEQ ID NO:8, (2) MAGE-A1 HLA class II binding peptides, and (3) MAGE-A1 HLA class I or class II binding peptide of a non-MAGE-A1 tumor antigen, in an amount sufficient to ameliorate the disorder.

15. The method of claim 12, wherein the MAGE-A1 HLA-B44 binding peptide is selected from the group consisting of (i) peptides which consist of a fragment of the amino acid sequence of SEQ ID NO:2, (ii) peptides which comprise the amino acid sequence of SEQ ID NO:14, and (iii) functional variants of the peptides of (i) and (ii).

16. The method of claim 15, further comprising administering to the subject at least one isolated HLA binding peptide selected from the group consisting of (1) MAGE-A1 HLA class I binding peptides other than peptides comprising SEQ ID NO:14, (2) MAGE-A1 HLA class II binding peptides, and (3) MAGE-A1 HLA class I or class II binding peptide of a non-MAGE-A1 tumor antigen, in an amount sufficient to ameliorate the disorder.

17. A method for treating a subject, typed as HLA-B35 or HLA-B44 positive, having a disorder characterized by expression of MAGE-A1, comprising:
administering to the subject an amount of an agent which enriches selectively in the subject the presence of complexes of a HLA-B35 molecule and a MAGE-A1 HLA-B35 binding peptide comprising SEQ ID NO:10, or a HLA-B44 molecule and a MAGE-A1 HLA-B44 binding peptide comprising SEQ ID NO:53, in an amount sufficient to ameliorate the disorder.

18. The method of claim 17, wherein the agent comprises a MAGE-A1 HLA-B35 binding peptide selected from the group consisting of (i) peptides which consist of a fragment of the amino acid sequence of SEQ ID NO:2, (ii) peptides which comprise the amino acid sequence of SEQ ID NO:8, and (iii) functional variants of the peptides of (i) and (ii).

19. The method of claim 17, wherein the agent comprises a MAGE-A1 HLA-B44 binding peptide selected from the group consisting of (i) peptides which consist of a fragment of the amino acid sequence of SEQ ID NO:2, (ii) peptides which comprise the amino acid sequence of SEQ ID NO:14, and (iii) functional variants of the peptides of (i) and (ii).

20. A method for treating a subject, typed as HLA-B35 or HLA-B44 positive, having a disorder characterized by expression of MAGE-A1, comprising:
administering to the subject an amount of autologous T lymphocytes sufficient to ameliorate the disorder, wherein the T lymphocytes are specific for complexes of a HLA-B35 molecule and a MAGE-A1 HLA-B35 binding peptide comprising SEQ ID NO:10, or a HLA-B44 molecule and a MAGE-A1 HLA-B44 binding peptide comprising SEQ ID NO:53.

21. The method of claim 20 wherein the MAGE-A1 HLA-B35 binding peptide is selected from the group consisting of (i) peptides which consist of a fragment of the amino acid sequence of SEQ ID NO:2, (ii) peptides which comprise the amino acid sequence of SEQ ID NO:8, and (iii) functional variants of the peptides of (i) and (ii).

22. The method of claim 20 wherein the MAGE-A1 HLA-B44 binding peptide is selected from the group consisting of (i) peptides which consist of a fragment of the amino acid sequence of SEQ ID NO:2, (ii) peptides which comprise the amino acid sequence of SEQ ID NO:14, and (iii) functional variants of the peptides of (i) and (ii).

23. A method for identifying functional variants of a MAGE-A1 HLA-B35 or HLA-B44 binding peptide, comprising
providing (1) a MAGE-A1 HLA-B35 binding peptide comprising SEQ ID NO:10, and a HLA-B35 molecule, or (2) a MAGE-A1 HLA-B44 binding peptide comprising SEQ ID NO:53, and a HLA-B44 molecule, and a T cell which is stimulated by the MAGE-A1 HLA binding peptide presented by the HLA-B35 or HLA-B44 molecule;
mutating a first amino acid residue of the MAGE-A1 HLA-B35 or HLA-B44 binding peptide to prepare a variant peptide; and
determining the binding of the variant peptide to the HLA-B35 or HLA-B44 molecule or the stimulation of the T cell, wherein binding of the variant peptide to the HLA binding molecule or stimulation of the T cell by the variant peptide presented by the HLA binding molecule indicates that the variant peptide is a functional variant.

24. The method of claim 23, wherein the MAGE-A1 HLA-B35 binding peptide is selected from the group consisting of (i) peptides which consist of a fragment of the amino acid sequence of SEQ ID NO:2, (ii) peptides which comprise the amino acid sequence of SEQ ID NO:8.

25. The method of claim 23, wherein the MAGE-A1 HLA-B44 binding peptide is selected from the group consisting of (i) peptides which consist of a fragment of the amino acid sequence of SEQ ID NO:2, (ii) peptides which comprise the amino acid sequence of SEQ ID NO:14.

26. The method of claim 23, further comprising the step of comparing the stimulation of the T cell by the MAGE-A1 HLA binding peptide and the stimulation of the T cell by the functional variant as a determination of the effectiveness of the stimulation of the T cell by the functional variant.

27. An expression vector comprising a nucleotide sequence which encodes an amino acid sequence selected from the group consisting of SEQ ID NO:8, SEQ ID NO:9, and SEQ ID NO:10, operably linked to a promoter, and a nucleotide sequence which encodes the amino acid sequence of a HLA-B*35 molecule, operably linked to a promoter.

28. An expression vector comprising a nucleotide sequence which encodes an amino acid sequence selected from the group consisting of SEQ ID NO:12, SEQ ID NO:14 and SEQ ID NO:53, operably linked to a promoter, and a nucleotide sequence which encodes the amino acid sequence of a HLA-B*44 molecule operably linked to a promoter.

29. A host cell transfected or transformed with the expression vector of claim 27.

30. A host cell transfected or transformed with the expression vector of claim 28.

31. An isolated T lymphocyte which selectively binds a complex of a HLA-B35 molecule and a MAGE-A1 HLA binding peptide which comprises the amino acid sequence of SEQ ID NO:10.

32. The isolated T lymphocyte of claim 31 wherein the MAGE-A1 HLA binding peptide is selected from the group consisting of (i) peptides which consist of a fragment of the amino acid sequence of SEQ ID NO:2, (ii) peptides which comprise the amino acid sequence of SEQ ID NO:8, and (iii) functional variants of the peptides of (i) and (ii).

33. An isolated T lymphocyte which selectively binds a complex of a HLA-B44 molecule and a MAGE-A1 HLA binding peptide which comprises the amino acid sequence of SEQ ID NO:53.

34. The isolated T lymphocyte of claim 33 wherein the MAGE-A1 HLA binding peptide is selected from the group consisting of (i) peptides which consist of a fragment of the amino acid sequence of SEQ ID NO:2, (ii) peptides which comprise the amino acid sequence of SEQ ID NO:14, and (iii) functional variants of the peptides of (i) and (ii).

35. An isolated antigen presenting cell which comprises a complex of a HLA-B35 molecule and a MAGE-A1 HLA binding peptide which comprises the amino acid sequence of SEQ ID NO:10.

36. The isolated antigen presenting cell of claim 35 wherein the MAGE-A1 HLA binding peptide is selected from the group consisting of (i) peptides which consist of a fragment of the amino acid sequence of SEQ ID NO:2, (ii) peptides which comprise the amino acid sequence of SEQ ID NO:8, and (iii) functional variants of the peptides of (i) and (ii).

37. An isolated antigen presenting cell which comprises a complex of a HLA-B44 molecule and a MAGE-A1 HLA binding peptide which comprises the amino acid sequence of SEQ ID NO:53.

38. The isolated antigen presenting cell of claim 37 wherein the MAGE-A1 HLA binding peptide is selected from the group consisting of (i) peptides which consist of a fragment of the amino acid sequence of SEQ ID NO:2, (ii) peptides which comprise the amino acid sequence of SEQ ID NO:14, and (iii) functional variants of the peptides of (i) and (ii).

39. A vaccine composition comprising a cell selected from the group consisting of a T lymphocyte of claims 31-34 and an antigen presenting cell of claims 35-38, and a pharmaceutically acceptable carrier.

40. The vaccine composition of claim 39, further comprising an adjuvant.

41. An isolated functional variant of a MAGE-A1 HLA-B35 or HLA-B44 binding peptide identified by the method of claim 23.

42. A method for identifying a candidate mimetic of a MAGE-A1 HLA-B35 or HLA-B44 binding peptide, comprising
providing a HLA-B35 or HLA-B44 molecule,
contacting the HLA molecule with a test molecule, and
determining the binding of the test molecule to the HLA molecule, wherein a test molecule which binds to the HLA molecule is a candidate mimetic of the MAGE-A1 HLA binding peptide.

43. The method of claim 42, further comprising contacting the HLA molecule with a HLA-B35 binding molecule comprising the amino acid sequence of SEQ ID NO:8, and determining the binding of the HLA binding molecule to the HLA molecule in the presence and the absence of the test molecule.

44. The method of claim 42, further comprising contacting the HLA molecule with a HLA-B44 binding molecule comprising the amino acid sequence of SEQ ID NO:53, and determining the binding of the HLA binding molecule to the HLA molecule in the presence and the absence of the test molecule.

45. The method of claim 42, further comprising
forming a complex of the HLA molecule and the candidate mimetic,
contacting the complex with a T cell which binds to a complex of a HLA molecule and the MAGE-A1 HLA binding peptide, and
assaying activation of the T cell.

46. The method of claim 45, wherein activation of the T cell is indicated by a property selected from the group consisting of proliferation of the T cell, interferon-γ production by the T cell, tumor necrosis factor production by the T cell, and cytolysis of a target cell by the T cell.

47. An isolated mimetic of a MAGE-A1 HLA-B35 or HLA-B44 binding peptide identified by the method of claim 42.

48. A method for diagnosing a disorder characterized by expression of MAGE-A3 in a subject typed as HLA-B35 positive, comprising:
contacting a biological sample isolated from the subject with an agent that is specific for a MAGE-A3 HLA binding peptide which comprises SEQ ID NO:56, and
determining the interaction between the agent and the MAGE-A3 HLA binding peptide as a determination of the disorder.

49. The method of claim 48 wherein the MAGE-A3 HLA binding peptide is selected from the group consisting of (i) peptides which consist of a fragment of the amino acid sequence of SEQ ID NO:55, (ii) peptides which comprise the amino acid sequence of SEQ ID NO:59, and (iii) functional variants of the peptides of (i) and (ii).

50. A method for diagnosing a disorder characterized by expression of a complex of a MAGE-A3 HLA-B35 binding peptide and a HLA-B35 molecule, comprising:
contacting a biological sample isolated from a subject suspected of having the disorder with an agent that binds the complex; and
determining binding between the complex and the agent as a determination of the disorder.

51. The method of claim 50 wherein the MAGE-A3 HLA-B35 binding peptide is selected from the group consisting (i) peptides which consist of a fragment of the amino acid sequence of SEQ ID NO:55, (ii) peptides which comprise the amino acid sequence of SEQ ID NO:56, and (iii) functional variants of the peptides of (i) and (ii).

52. A method for enriching selectively a population of T lymphocytes with T lymphocytes specific for a MAGE-A3 HLA binding peptide, comprising:
contacting a source of T lymphocytes which contains a population of T lymphocytes with an agent presenting a complex of a MAGE-A3 HLA-B35 binding peptide comprising SEQ ID NO:56 and a HLA-B35 molecule, in an amount sufficient to selectively enrich the population of T lymphocytes with the T lymphocytes specific for one of the complexes.

53. The method of claim 52, wherein the agent is an antigen presenting cell which expresses a HLA-B35 molecule contacted with a MAGE-A3 protein or a HLA binding fragment thereof which comprises SEQ ID NO:56.

54. The method of claim 52 wherein the MAGE-A3 HLA-B35 binding peptide is selected from the group consisting of (i) SEQ ID NO:56, SEQ ID NO:57 and SEQ ID NO:59, and (ii) functional variants of the peptides of (i).

55. A method for treating a subject, typed as HLA-B35 positive, having a disorder characterized by expression of MAGE-A3, comprising:
administering to the subject an amount of a MAGE-A3 HLA-B35 binding peptide comprising SEQ ID NO:56 sufficient to ameliorate the disorder.

56. The method of claim 55, wherein the MAGE-A3 HLA-B35 binding peptide is selected from the group consisting of (i) peptides which consist of a fragment of the amino acid sequence of SEQ ID NO:55, (ii) peptides which comprise the amino acid sequence of SEQ ID NO:59, and (iii) functional variants of the peptides of (i) and (ii).

57. The method of claim 55, further comprising administering to the subject at least one isolated HLA binding peptide selected from the group consisting of (1) MAGE-A3 HLA class I binding peptides other than peptides comprising SEQ ID NO:59, (2) MAGE-A3 HLA class II binding peptides, and (3) HLA class I or class II binding peptide of a non-MAGE-A3 tumor antigen, in an amount sufficient to ameliorate the disorder.

58. A method for treating a subject, typed as HLA-B35 positive, having a disorder characterized by expression of MAGE-A3, comprising:
administering to the subject an amount of an agent which enriches selectively in the subject the presence of complexes of a HLA-B35 molecule and a MAGE-A3 HLA-B35 binding peptide comprising SEQ ID NO:56, in an amount sufficient to ameliorate the disorder.

59. The method of claim 58, wherein the agent comprises a MAGE-A3 HLA-B35 binding peptide selected from the group consisting of (i) peptides which consist of a fragment of the amino acid sequence of SEQ ID NO:55, (ii) peptides which comprise the amino acid sequence of SEQ ID NO:59, and (iii) functional variants of the peptides of (i) and (ii).

60. A method for treating a subject, typed as HLA-B35 positive, having a disorder characterized by expression of MAGE-A3, comprising:
administering to the subject an amount of autologous T lymphocytes sufficient to ameliorate the disorder, wherein the T lymphocytes are specific for complexes of a HLA-B35 molecule and a MAGE-A3 HLA-B35 binding peptide comprising SEQ ID NO:56.

61. The method of claim 60 wherein the MAGE-A3 HLA-B35 binding peptide is selected from the group consisting of (i) peptides which consist of a fragment of the amino acid sequence of SEQ ID NO:55, (ii) peptides which comprise the amino acid sequence of SEQ ID NO:59, and (iii) functional variants of the peptides of (i) and (ii).

62. A method for identifying functional variants of a MAGE-A3 HLA-B35 binding peptide, comprising providing a MAGE-A3 HLA-B35 binding peptide comprising SEQ ID NO:56, a HLA-B35 molecule, and a T cell which is stimulated by the MAGE-A3 HLA binding peptide presented by the HLA-B35 molecule;
mutating a first amino acid residue of the MAGE-A3 HLA-B35 binding peptide to prepare a variant peptide; and
determining the binding of the variant peptide to the HLA-B35 molecule or the stimulation of the T cell, wherein binding of the variant peptide to the HLA binding molecule or stimulation of the T cell by the variant peptide presented by the HLA binding molecule indicates that the variant peptide is a functional variant.

63. The method of claim 62, wherein the MAGE-A3 HLA-B35 binding peptide is selected from the group consisting of (i) peptides which consist of a fragment of the amino acid sequence of SEQ ID NO:55, (ii) peptides which comprise the amino acid sequence of SEQ ID NO:59.

64. The method of claim 62, further comprising the step of comparing the stimulation of the T cell by the MAGE-A3 HLA binding peptide and the stimulation of the T cell by the functional variant as a determination of the effectiveness of the stimulation of the T cell by the functional variant.

65. An expression vector comprising a nucleotide sequence which encodes an amino acid sequence selected from the group consisting of SEQ ID NO:56, SEQ ID NO:57 and SEQ ID NO:59, operably linked to a promoter, and a nucleotide sequence which encodes the amino acid sequence of a HLA-B35 molecule, operably linked to a promoter.

66. A host cell transfected or transformed with the expression vector of claim 65.

67. An isolated T lymphocyte which selectively binds a complex of a HLA-B35 molecule and a MAGE-A3 HLA binding peptide which comprises the amino acid sequence of SEQ ID NO:56.

68. The isolated T lymphocyte of claim 67 wherein the MAGE-A3 HLA binding peptide is selected from the group consisting of (i) peptides which consist of a fragment of the amino acid sequence of SEQ ID NO:55, (ii) peptides which comprise the amino acid sequence of SEQ ID NO:59, and (iii) functional variants of the peptides of (i) and (ii).

69. An isolated antigen presenting cell which comprises a complex of a HLA-B35 molecule and a MAGE-A3 HLA binding peptide which comprises the amino acid sequence of SEQ ID NO:56.

70. The isolated antigen presenting cell of claim 69 wherein the MAGE-A3 HLA binding peptide is selected from the group consisting of (i) peptides which consist of a fragment of the amino acid sequence of SEQ ID NO:55, (ii) peptides which comprise the amino acid sequence of SEQ ID NO:59, and (iii) functional variants of the peptides of (i) and (ii).

71. A vaccine composition comprising a cell selected from the group consisting of a T lymphocyte of claims 67-68 and an antigen presenting cell of claims 69-70, and a pharmaceutically acceptable carrier.

72. The vaccine composition of claim 71, further comprising an adjuvant.

73. An isolated functional variant of a MAGE-A3 HLA-B35 binding peptide identified by the method of claim 62.

74. A method for identifying a candidate mimetic of a MAGE-A3 HLA-B35 binding peptide, comprising
providing a HLA-B35 molecule,
contacting the HLA molecule with a test molecule, and
determining the binding of the test molecule to the HLA molecule, wherein a test molecule which binds to the HLA molecule is a candidate mimetic of the MAGE-A3 HLA binding peptide.

75. The method of claim 74, further comprising contacting the HLA molecule with a HLA-B35 binding molecule comprising the amino acid sequence of SEQ ID NO:56, and determining the binding of the HLA binding molecule to the HLA molecule in the presence and the absence of the test molecule.

76. The method of claim 74, further comprising
forming a complex of the HLA molecule and the candidate mimetic,
contacting the complex with a T cell which binds to a complex of a HLA molecule and the MAGE-A3 HLA binding peptide, and
assaying activation of the T cell.

77. The method of claim 76, wherein activation of the T cell is indicated by a property selected from the group consisting of proliferation of the T cell, interferon-γ production by the T cell, tumor necrosis factor production by the T cell, and cytolysis of a target cell by the T cell.

78. An isolated mimetic of a MAGE-A3 HLA-B35 binding peptide identified by the method of claim 74.


SEQUENCE LISTING (59 sequences)

SEQ ID NO: 1 - DNA, 930 bp, Homo sapiens
atgtctcttgagcagaggagtctgcactgcaagcctgaggaagcccttgaggcccaacaagaggccctgggcctggtgtgtgtgcaggctgccacctcctcctcctctcctctggtcctgggcaccctggaggaggtgcccactgctgggtcaacagatcctccccagagtcctcagggagcctccgcctttcccactaccatcaacttcactcgacagaggcaacccagtgagggttccagcagccgtgaagaggaggggccaagcacctcttgtatcctggagtccttgttccgagcagtaatcactaagaaggtggctgatttggttggttttctgctcctcaaatatcgagccagggagccagtcacaaaggcagaaatgctggagagtgtcatcaaaaattacaagcactgttttcctgagatcttcggcaaagcctctgagtccttgcagctggtctttggcattgacgtgaaggaagcagaccccaccggccactcctatgtccttgtcacctgcctaggtctctcctatgatggcctgctgggtgataatcagatcatgcccaagacaggcttcctgataattgtcctggtcatgattgcaatggagggcggccatgctcctgaggaggaaatctgggaggagctgagtgtgatggaggtgtatgatgggagggagcacagtgcctatggggagcccaggaagctgctcacccaagatttggtgcaggaaaagtacctggagtaccggcaggtgccggacagtgatcccgcacgctatgagttcctgtggggtccaagggccctcgctgaaaccagctatgtgaaagtccttgagtatgtgatcaaggtcagtgcaagagttcgctttttcttcccatccctgcgtgaagcagctttgagagaggaggaagagggagtctga

SEQ ID NO: 2 - protein, 309 aa, Homo sapiens
MSLEQRSLHCKPEEALEAQQEALGLVCVQAATSSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGV

SEQ ID NO: 3 - DNA, 31 bp, Homo sapiens
aaactgcagatgtctcttgagcagaggagtc

SEQ ID NO: 4 - DNA, 30 bp, Homo sapiens
aaactgcagtcagactccctcttcctcctc

SEQ ID NO: 5 - protein, 12 aa, Homo sapiens
EADPTGHSYVLV

SEQ ID NO: 6 - protein, 10 aa, Homo sapiens
DPTGHSYVLV

SEQ ID NO: 7 - protein, 9 aa, Homo sapiens
DPTGHSYVL

SEQ ID NO: 8 - protein, 9 aa, Homo sapiens
EADPTGHSY

SEQ ID NO: 9 - protein, 10 aa, Homo sapiens
KEADPTGHSY

SEQ ID NO: 10 - protein, 8 aa, Homo sapiens
ADPTGHSY

SEQ ID NO: 11 - DNA, 72 bp, Homo sapiens
atgtctgagtccttgcagctggtctttggcattgacgtgaaggaagcagaccccaccggccactcctattga

SEQ ID NO: 12 - protein, 23 aa, Homo sapiens
MSESLQLVFGIDVKEADPTGHSY

SEQ ID NO: 13 - DNA, 33 bp, Homo sapiens
atggaagcagaccccaccggccactcctattga

SEQ ID NO: 14 - protein, 10 aa, Homo sapiens
MEADPTGHSY

SEQ ID NO: 15 - DNA, 30 bp, Homo sapiens
atggcagaccccaccggccactcctattga

SEQ ID NO: 16 - protein, 9 aa, Homo sapiens
MADPTGHSY

SEQ ID NO: 17 - protein, 9 aa, Homo sapiens
SAYGEPRKL

SEQ ID NO: 18 - protein, 9 aa, Homo sapiens
EVDPIGHLY

SEQ ID NO: 19 - protein, 9 aa, Homo sapiens
FLWGPRALV

SEQ ID NO: 20 - protein, 10 aa, Homo sapiens
MEVDPIGHLY

SEQ ID NO: 21 - protein, 9 aa, Homo sapiens
AARAVFLAL

SEQ ID NO: 22 - protein, 8 aa, Homo sapiens
YRPRPRRY

SEQ ID NO: 23 - protein, 10 aa, Homo sapiens
SPSSNRIRNT

SEQ ID NO: 24 - protein, 9 aa, Homo sapiens
VLPDVFIRC

SEQ ID NO: 25 - protein, 10 aa, Homo sapiens
VLPDVFIRCV

SEQ ID NO: 26 - protein, 9 aa, Homo sapiens
EEKLIVVLF

SEQ ID NO: 27 - protein, 9 aa, Homo sapiens
EEKLSVVLF

SEQ ID NO: 28 - protein, 10 aa, Homo sapiens
ACDPHSGHFV

SEQ ID NO: 29 - protein, 10 aa, Homo sapiens
ARDPHSGHFV

SEQ ID NO: 30 - protein, 9 aa, Homo sapiens
SYLDSGIHF

SEQ ID NO: 31 - protein, 9 aa, Homo sapiens
SYLDSGIHS

SEQ ID NO: 32 - protein, 9 aa, Homo sapiens
MLLAVLYCL

SEQ ID NO: 33 - protein, 9 aa, Homo sapiens
YMNGTMSQV

SEQ ID NO: 34 - protein, 9 aa, Homo sapiens
AFLPWHRLF

SEQ ID NO: 35 - protein, 9 aa, Homo sapiens
SEIWRDIDF

SEQ ID NO: 36 - protein, 9 aa, Homo sapiens
YEIWRDIDF

SEQ ID NO: 37 - protein, 15 aa, Homo sapiens
QNILLSNAPLGPQFP

SEQ ID NO: 38 - protein, 15 aa, Homo sapiens
DYSYLQDSDPDSFQD

SEQ ID NO: 39 - protein, 10 aa, Homo sapiens
EAAGIGILTV

SEQ ID NO: 40 - protein, 9 aa, Homo sapiens
AAGIGILTV

SEQ ID NO: 41 - protein, 9 aa, Homo sapiens
ILTVILGVL

SEQ ID NO: 42 - protein, 9 aa, Homo sapiens
KTWGQYWQV

SEQ ID NO: 43 - protein, 9 aa, Homo sapiens
ITDQVPFSV

SEQ ID NO: 44 - protein, 9 aa, Homo sapiens
YLEPGPVTA

SEQ ID NO: 45 - protein, 10 aa, Homo sapiens
LLDGTATLRL

SEQ ID NO: 46 - protein, 10 aa, Homo sapiens
VLYRYGSFSV

SEQ ID NO: 47 - protein, 9 aa, Homo sapiens
LYVDSLFFL

SEQ ID NO: 48 - protein, 12 aa, Homo sapiens
KISGGPRISYPL

SEQ ID NO: 49 - protein, 9 aa, Homo sapiens
YMDGTMSQV

SEQ ID NO: 50 - protein, 11 aa, Homo sapiens
SLLMWITQCFL

SEQ ID NO: 51 - protein, 9 aa, Homo sapiens
SLLMWITQC

SEQ ID NO: 52 - protein, 9 aa, Homo sapiens
QLSLLMWIT

SEQ ID NO: 53 - protein, 10 aa, Homo sapiens
  UNSURE (1)..(1): Xaa = any amino acid
XEADPTGHSY

SEQ ID NO: 54 - DNA, 945 bp, Homo sapiens
atgcctcttgagcagaggagtcagcactgcaagcctgaagaaggccttgaggcccgaggagaggccctgggcctggtgggtgcgcaggctcctgctactgaggagcaggaggctgcctcctcctcttctactctagttgaagtcaccctgggggaggtgcctgctgccgagtcaccagatcctccccagagtcctcagggagcctccagcctccccactaccatgaactaccctctctggagccaatcctatgaggactccagcaaccaagaagaggaggggccaagcaccttccctgacctggagtccgagttccaagcagcactcagtaggaaggtggccgagttggttcattttctgctcctcaagtatcgagccagggagccggtcacaaaggcagaaatgctggggagtgtcgtcggaaattggcagtatttctttcctgtgatcttcagcaaagcttccagttccttgcagctggtctttggcatcgagctgatggaagtggaccccatcggccacttgtacatctttgccacctgcctgggcctctcctacgatggcctgctgggtgacaatcagatcatgcccaaggcaggcctcctgataatcgtcctggccataatcgcaagagagggcgactgtgcccctgaggagaaaatctgggaggagctgagtgtgttagaggtgtttgaggggagggaagacagtatcttgggggatcccaagaagctgctcacccaacatttcgtgcaggaaaactacctggagtaccggcaggtccccggcagtgatcctgcatgttatgaattcctgtggggtccaagggccctcgttgaaaccagctatgtgaaagtcctgcaccatatggtaaagatcagtggaggacctcacatttcctacccacccctgcatgagtgggttttgagagagggggaagagtga

SEQ ID NO: 55 - protein, 314 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 56 - protein, 9 aa, Homo sapiens
EVDPIGHLY

SEQ ID NO: 57 - protein, 16 aa, Homo sapiens
MEVDPIGHLYIFACTL

SEQ ID NO: 58 - protein, 9 aa, Homo sapiens
DPIGHLYIF

SEQ ID NO: 59 - protein, 10 aa, Homo sapiens
MEVDPIGHLY